(19) United States
(12) Patent Application Publication
(10) Pub. No.: US 2012/0029832 A1
(43) Pub. Date: Feb. 2, 2012

(54) Apparatus and method for distribution of biological material

(76) Inventor: John R. Dodgson, London (GB)

(21) Appl. No.: 13/191,228

(22) Filed: Jul. 26, 2011

Related U.S. Application Data
(60) Provisional application No. 61/367,943, filed on Jul. 27, 2010.

(51) Int. Cl. G01N 33/48; G06F 19/00

(52) U.S. Cl. 702/19

(57) ABSTRACT

A new approach is proposed that contemplates systems and methods to support improved means for handling of biological materials, for example biological samples or products used in biological or biomedical research, diagnostics or therapy. In particular, the proposed approach provides improved means for handling patient samples and products used in regenerative medicine, such as biological or biochemical products or cell products. Improvements in for example, the ability to culture, process, distribute or preserve, i.e. increase the usable life of biological products, including cell products, in regenerative medicine would enable more widespread use of such products and uptake of regenerative medicine procedures.

### RELATED APPLICATIONS

This application claims the benefit of U.S. Provisional Patent Application No. 61/367,943, filed Jul. 27, 2010, and entitled “Apparatus and Method for Distribution of Biological Material,” and is hereby incorporated herein by reference.

### FIELD OF THE INVENTION

This invention relates to apparatus, a system and method for handling, for example culturing, processing, transporting or preserving biological or biochemical materials, which may be cellular materials such as cell cultures, cell suspensions, biological tissue, biosamples, as used for example in the field of regenerative medicine or cell therapy.

### BACKGROUND

There is a need for improved means for handling of biological materials, for example biological samples or products used in biological or biomedical research, diagnostics or therapy. In particular, there is a need for improved means for handling patient samples and products used in regenerative medicine, such as biological or biochemical products or cell products. Improvements in for example, the ability to culture, process, distribute or preserve, i.e. increase the usable life of biological products, including cell products, in regenerative medicine would enable more widespread use of such products and uptake of regenerative medicine procedures.

### BRIEF DESCRIPTION OF THE DRAWINGS

FIG. 1 shows a diagrammatic view of an embodiment of an apparatus according to the invention

FIG. 2a shows a diagrammatic view of a further embodiment of an apparatus according to the invention.

FIG. 2b shows a diagrammatic view of a further embodiment of an apparatus according to the invention.

FIG. 2c shows a diagrammatic view of a further embodiment of an apparatus according to the invention, illustrating multiple stages in a process according to a method of the invention.

FIGS. 3a-3j together show a diagrammatic view of a further embodiment of an apparatus according to the invention. FIGS. 3a-3j each show a stage in a multistage process according to the method of the invention.

FIG. 4a shows a cross-sectional diagram of a further embodiment of an apparatus according to the invention

FIG. 4b shows a cross-sectional diagram of a further embodiment of an apparatus according to the invention

FIG. 4c shows a cross-sectional diagram of a further embodiment of an apparatus according to the invention with a movable component in a second position

FIG. 4d shows a cross-sectional diagram of the embodiment shown in FIG. 14c having the movable component in a first position

FIG. 4e shows an external elevation view of the embodiment shown in FIG. 14c

FIGS. 5a-5d show cross-sectional diagrams of further embodiments of apparatus according to the invention

FIG. 6 shows a cross-sectional diagram of a further embodiment of an apparatus according to the invention

FIG. 7a shows a cross section of a first embodiment of an apparatus according to the invention

FIG. 7b shows a cross section of a further embodiment of an apparatus according to the invention

FIG. 8 shows a cross section of a further embodiment of an apparatus according to the invention

FIG. 9 shows a cross section of a further embodiment of an apparatus according to the invention

FIG. 10 shows a three-quarter view of a further embodiment of an apparatus according to the invention

FIG. 11 shows a cross sectional view of the embodiment of FIG. 10

FIG. 12 shows a cross section of a further embodiment of an apparatus according to the invention

FIG. 13 shows a three-quarter view of a first component of a further embodiment of an apparatus according to the invention

FIG. 14 shows a three-quarter view of a first later stage of the component shown in FIG. 13 following a first step in a method according to the invention

FIG. 15 shows a three-quarter view of a second later stage of the component shown in FIG. 13 following a further step in a method according to the invention

FIG. 16 shows a three-quarter view of a third later stage of the component shown in FIG. 13 following a further step in a method according to the invention.

FIG. 17 shows a diagrammatic view of an apparatus according to the invention in operation together with a component apparatus of the invention.

### DETAILED DESCRIPTION OF EMBODIMENTS OF THE INVENTION

According to the invention are provided apparatus and a method to achieve improvements in processes including the following:

Handling of cellular products outside a production facility allowing distribution of the products to patients with increased usable life of the products.

Handling of biological materials, such as patient samples, processed material from such samples, and cell products, using closed system interfaces between stages of the handling process, so minimising risks to contamination.

According to a first aspect the invention provides an apparatus, a system and method for processing and/or distribution of such products while gathering information on the process automatically, so reducing the administrative effort needed to monitor the process for example for regulatory purposes.

Accordingly in an embodiment of the invention an apparatus and system is provided for enabling the processing and/or distribution of a biological material, such as for example a product used in regenerative medicine, regarding the progress of the process or distribution, collecting and collating information usable in the treatment of a patient or the reporting of parameters for regulatory purposes.

It is a feature that the invention is adapted to make such data gathering and collation routine and to an extent automatic, saving cost in data gathering and reporting and enabling more effective regulation of regenerative medicine processes.

It is a further feature that the apparatus and method of the invention is adapted to enable ready communication of the data so gathered to an external data system, such as a Hospital Information System (HIS) or a Laboratory Information Management System (LIMS). For example, the apparatus of the system may be adapted to:

Gather regulatory compliance data automatically and compile reports

Gather and store process data as part of the HIS patient records.

Schedule patient treatment based on data from a regenerative medicine product production and distribution process, allowing effective use of clinical time.

Regulate the production and distribution process, based on clinical considerations such as availability of clinician and patient or the health status of the patient.

The apparatus of the invention is envisaged as having multiple components adapted to be interoperable one with another and which may be located in more than one physical location, being adapted to operate together by means of data links and in some embodiments the physical movement of device comprising data stored in memory associated with those devices. Therefore the apparatus of the invention is referred to on occasion as a ‘system’. References to ‘apparatus’ and ‘system’ are taken as interchangeable in this context, except where specifically stated otherwise.

According to a further embodiment the invention provides an apparatus and a system comprising one or more product having a closed system interface to one another or to one or more existing apparatus or devices used in production of a regenerative medicine product or administration of such products to a patient or the taking of a biopsy from a patient. Closed system interfaces are those that can be made and/or broken without compromising the sterility of the contents of an apparatus or device, or the pathway for a regenerative medicine product into or out from the apparatus or device.

According to a further embodiment the invention provides an apparatus and a system comprising two or more component apparatuses or devices that link together so as to form part of the process of production or distribution of a regenerative medicine product, that linkage preferably comprising a closed system interface through which the product or a component thereof may pass; and a data link that establishes a data association between data pertaining to a first component apparatus or device and that pertaining to a second. The data association may be in a data system remote from the component apparatus or devices, or may be local to them for example in the form of read/write data storage comprised within one or both components.

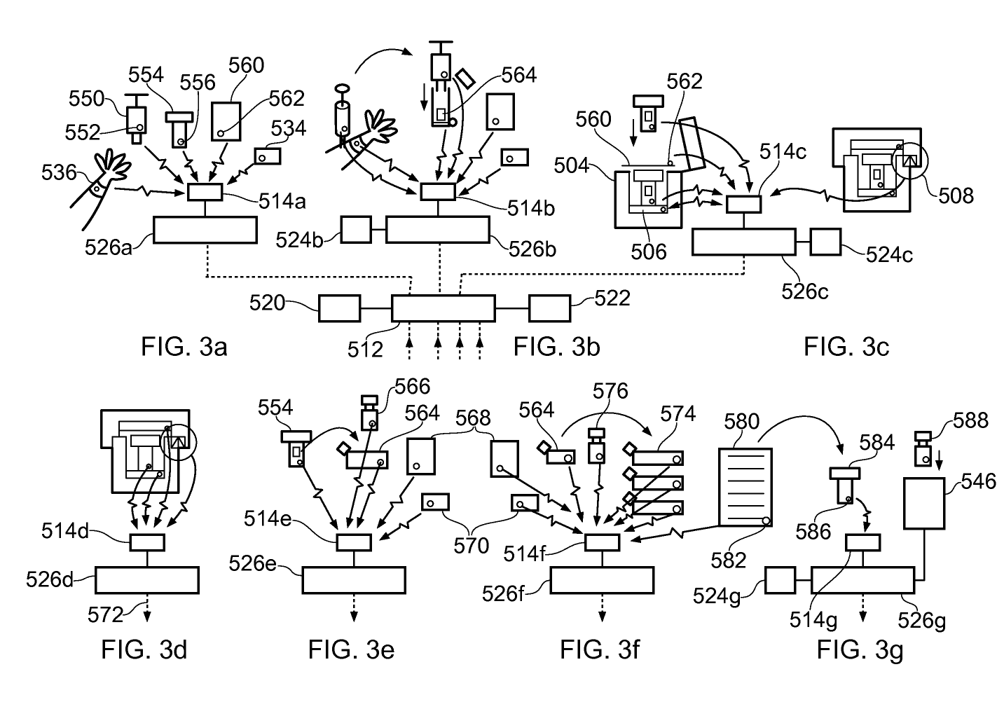

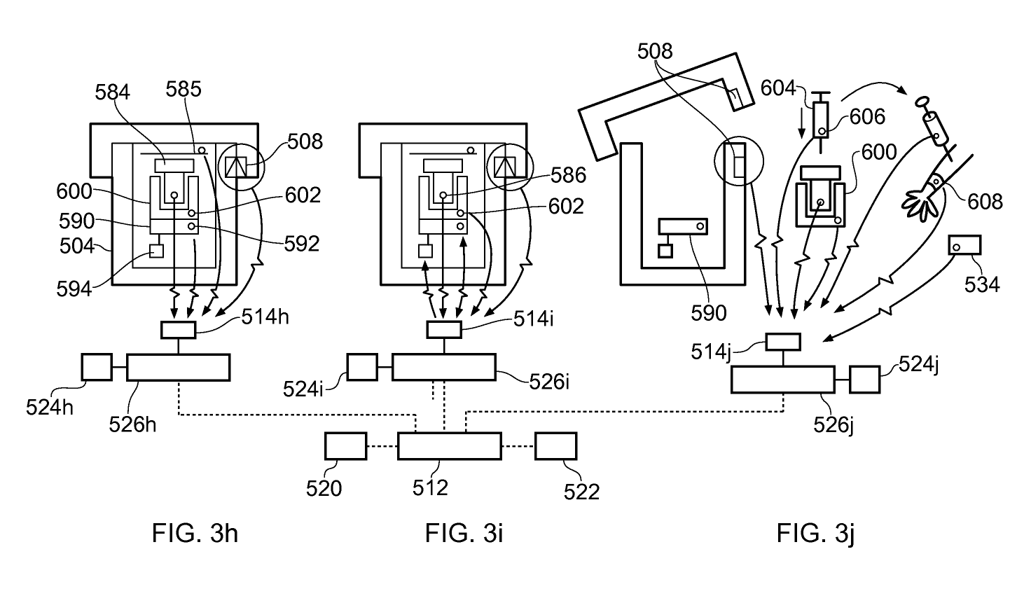

According to a further aspect of the invention is provided an apparatus and system for use in a diagnostic or treatment process such as an autologous or allogeneic regenerative medicine process, comprising one or more of the following components (referring to FIGS. 3a-3j, 4a, 7a and 7b):

Biopsy device (550)

Biopsy package (556)

Biopsy package transport carrier (504)

Biopsy processor adapted for use in isolation of a chosen component from the Biopsy

Cell processing devices such as cell culture devices (564, 574, 580) forming part of a production process for the Regenerative medicine product

Regenerative Medicine product distribution devices (584, 700, 10, 100)

Regenerative Medicine product distribution device transport carrier (504) (FIG. 3h)

Point of use processing apparatus for processing of the Regenerative Medicine product at the clinic (510)

Administration device to administer the Regenerative Medicine product to a patient

Data System that interacts with data tags associated with one or more of the components to track the stages of autologous regenerative medicine process and compile reports

According to a further aspect the invention provides a container for biological material comprising identification and monitoring means adapted to be used with the apparatus and system above, and a system comprising the container and a data system that in use may receive data from or send data to the monitoring means, in order to provide an improved means of monitoring a process of storage, transport or distribution of the biological material.

In an embodiment of invention is provided a cell product package adapted to contain a cell product comprising cells in suspension, further adapted to facilitate concentration of the cells leaving a supernatant liquid, further adapted to facilitate removal of the cell concentrate from the cell product package, for example into a patient administration device such as a syringe or catheter.

In further embodiments the invention provides apparatus, systems and methods for handling, for example culture, transport, processing or preservation of a biological material. The biological material may be a product intended for use in medicine, for example in regenerative medicine, and may be a cell product, namely a product containing living cells. In the following the terms ‘cell product’ and ‘biological material’ are used interchangeably except where specifically pointed out.

The biological material may comprise a regenerative medicine product, which may comprise as active ingredients cells, cytokines or other biologically active materials or mixtures thereof.

In a preferred embodiment the apparatus comprises a biological material distribution device which may comprise a biological material package, which may be adapted to contain a cell product. The terms ‘biological material package’ and ‘cell product package’ are used interchangeably throughout except where specifically pointed out.

In one aspect the invention provides an apparatus and system for the distribution of a biological material comprising:

One or more biological material distribution devices, each comprising a readable data tag having device information stored on it

A data system comprising one or more terminals adapted to communicate with the data tag to read the device information

A data program that receives data from the terminal(s) comprising information read from the data tag and analyses the data to produce results relating to the device information.

The data tag may have a unique code and may have further device information permanently stored thereon.

The data tag may have a read/write function and may have device information written to it during manufacture or preparation before use in the system, and may have data written by a terminal during use as part of the system.

The data tag may be an RFID (Radio Frequency Identification Device) tag, an optical printed tag such as a barcode or hologram, or an electronic system with radio or wired electronic, or active optical communication to a terminal.

The device information may comprise information relating to one or more processes through which the biological material distribution device has passed.

Such processes might include fabrication of the device, sterilisation, supply to the user, first entry into use, filling, checking, quality control processes, packaging, transporting, control of temperature or other environmental variables, such as gas atmosphere, arrival at a point of use, storage, entry into use, opening of packaging, use, discard, return shipping, disposal or recycling.

Device information stored on the tag might include: device batch number, serial number, fabrication date, product contents and information regarding these, usage instructions, logged history of use such as temperature, time, shock, gas environment, opening/sealing events.

Device information stored on the tag might include information on interaction with people such as process operators, shipping personnel, clinical staff, a patient, or with one or more other devices which form part of the system or with which the devices of the system are adapted to communicate.

The device information may comprise data regarding a patient, a treatment protocol, a member of clinical staff, for example who performs a protocol, a treatment site or other information specific to the diagnosis, investigation or treatment of a patient's indication.

The data system or data tag may interface with a Hospital Information System (HIS) to send or receive data, so transferring data from the HIS to the data tag or from the data tag to the HIS.

The data system or data tag may interface with a Laboratory Information Management System (LIMS) to send or receive data, so transferring data from the LIMS to the data tag or from the data tag to the LIMS. The LIMS might be for example the property of a Cell Therapy company, a cell product manufacturing company, such as a Contract Manufacturing Organisation (CMO), a Clinical Research Organisation (CRO) or a validation or regulatory organisation.

The data system may be adapted to provide information in a form that is readily usable by one or more such organisations above in written, printed, form or in electronic form.

In one embodiment the apparatus and system is adapted for distribution of a cell product, for example in use in cell therapy and may operate with one or more of the following steps, the invention in a further aspect providing a method comprising one or more of the steps:

A cell product distribution device (“device’) is manufactured and comprises a unique ID tag;

The device is filled with a cell product. The process data for that product are stored in a database forming part of the data system, and the device ID is associated in the database with the process data;

The device is packaged into a controlled environment shipper. The shipper may have a data tag comprising a unique ID, and the shipper ID may be read and associated in the database with the process data;

The shipper may have a temperature/time logger recording the environment of the device;

On receipt at a clinic, one or both data tags are read and arrival and other shipping data are associated with the process data in the database;

Temperature/time data may be downloaded and associated;

The package may be opened and used with a patient admin device. The admin device may have a tag compatible with the data system. The admin device tag ID may be read and associated with the process data in the database. Details of the treatment procedure may be recorded and communicated to the data system;

Process and other data may be communicated from the data system to the HIS, so adding to the patient record information about the process that the product has passed through on its way to the point of administration to the patient.

In this way the system of the invention may form part of an data tracking system that tracks and reports the overall therapy delivered to the patient, from the production process of the therapy, through distribution and arrival at the clinic, storage at the clinic and administration to the patient.

The system of the invention may further comprise one or more of the following, each comprising a data tag as above:

Means for taking a biopsy from a patient, such as a sample of fluid, blood, tissue, cells;

A biopsy package adapted to contain the biopsy;

A biopsy processing apparatus adapted to isolate a component of interest from the biopsy;

An interface unit to a production process, for example at a CMO;

Apparatus in use at one or more stages of the production process;

Apparatus in use in a quality control process either before, during or after the cell production process;

Apparatus used to treat, prepare, fill or seal a biological material distribution device;

Controlled environment shipping apparatus;

Apparatus for processing of the biological material at the clinic (‘point of care processing’ or ‘POC processing’);

Apparatus for quality control of the biological material at the clinic.

The system may comprise one, or multiple component apparatus or devices, and may comprise a data system that communicates with one or more of the component apparatus or devices.

The system may comprise one, or multiple terminals via which such communication may be mediated.

In a further embodiment, the system may comprise two or more component devices each equipped with a data tag, at least one of the data tags being adapted to read or write to or from one of the other tags within the system. In this way, data may be passed from a first component apparatus or device to a second component apparatus or device, optionally without communication to the data system.

The system may comprise multiple component apparatus and devices, two or more of which comprise a read/write data tag that is capable of passing data to, or receiving data from, another data tag in the system. Data may then be transferred directly from one component apparatus or device to a second, and then optionally to a third, etc. The data tags may send/receive data to/from the data system via a terminal at stages in the process. In this way, data may be transferred from the first component around a chain of component each forming part of the system. Data may be read from a tag to the HIS directly, or after processing via the data system.

A data tag may comprise RFID data tags as known in the art, such as a read only unique ID tag; a read/write tag with on-tag data storage capability; a tag comprising sensors such as temperature, shock, humidity, gas concentration; a GPS localisation device. They may be powered or unpowered.

The data tag may be built into the component apparatus or device or may be attached or associated with it. In the case of a data tag associated with a distribution device comprising a fluid product, the data tag might be reversibly attached to the distribution device. The data tag might be re-usable with further distribution devices or may be an integral part of the distribution device and disposable with it.

In a further aspect the invention provides a method of processing or distributing a Regenerative medicine product comprising one or more of the steps of:

Providing a Regenerative medicine product processing or distribution system as described above

Using one or more of the components of the system to contain, process, distribute or modify a Regenerative medicine product

Causing data to be transferred from one or more of the components of the system to a second component or a data system forming part of the System

Producing a report using that data

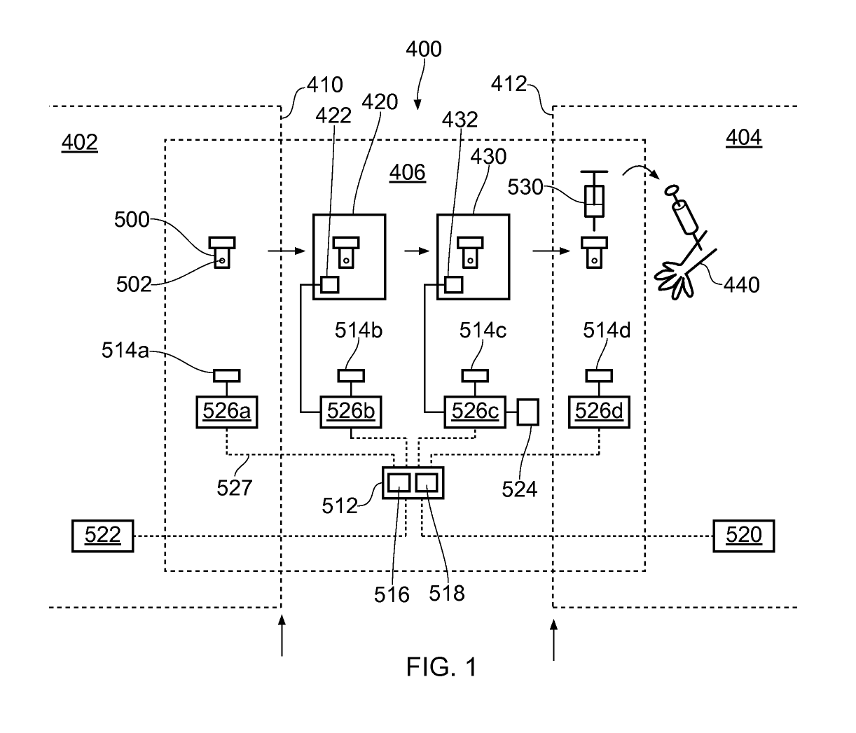

In a preferred embodiment the apparatus of the invention is adapted to monitor a multi-stage process of handling a biological material and to determine whether the overall process meets requirements for regulatory compliance. In a further embodiment the apparatus of the invention provides a means for processing a biological material through a multiple stage process while gathering data from the process and using the data to determine whether the process meets requirements for regulatory compliance. FIG. 1 shows an apparatus according to the invention adapted to handle a biological material and to monitor parameters of one or more process stages forming part of an overall process through which a biological material passes, and then to determine whether the overall process formed by the sum of such process stages meets the requirements for regulatory compliance. An apparatus 400 comprises a biological material handling device 500 having a tag 502 that holds information relating to the device or its contents, i.e. ‘device information’, comprising at least an ID code for the device. The apparatus further comprises one or more component apparatus 420, 430 adapted to carry out a process on the on the biological material, each comprising a sensor means 422, 432 to monitor a condition associated with the biological material handling device during the processing, and a data system comprising one or more subsystems each comprising terminal means 514a-514d, adapted to read data from the data tag 502, and control means 526a-526d having a data linkage to the component apparatus to receive data from the sensor means; a processing unit 512, such as a server computer, comprising a database 516 and a data analysis means such as a data program 518, the database being adapted to store and associate data read from the tag and from the sensors related to the processing of the biological material and the data analysis means being adapted to analyse the data and generate a report on regulatory compliance of the process and/or of the biological material. The apparatus may then indicate compliance or non-compliance at any point in the process, in a preferred embodiment at the point of care or immediately after the last stage of the process before the point of care. Indicator means 524 may be provided as a ‘go/no-go’ indication on the compliance of the process or material.

In a preferred embodiment apparatus 400 is in the form of a system comprising component apparatus distributed in more than one location, the apparatus comprising means for exchange of data between them, such as a data network linking the data system and the component apparatus as shown by dotted lines 527. The data system preferably also has data linkages to all exchange of information with one or both of a Hospital Information System (HIS) 520 and a Laboratory Information Management System (LIMS) 522.

In use the apparatus of the invention may provide an interface between a first regulatory environment controlling the production of a biological material, shown as the area 402 within the dotted line in FIG. 1, and a second regulatory environment controlling its use at the clinic, shown as area 404. The apparatus and method of the invention may be applied to provide a regulatory environment 406 that acts as a bridge between the two, allowing regulatory oversight of the product during the processes between the first regulatory environment and the second. In use boundary 410 between the production environment and the environment 406 and boundary 412 between environment 406 and the clinical environment 404 may be regarded as stage gates through which the biological material passes. The biological material passes a QC process in the production environment and enters the environment 406 that is controlled and monitored by the apparatus and method. The data system monitors the processes through which the biological material passes and analyses these for compliance. The biological material may then be passed through the second stage gate 412 if the processes have been compliant and the biological material may then be administered to a patient 440 using an administration means 530. Components of the data system may be provided within the product regulatory environment 514a, 526a and within the clinical regulatory environment 514d, 526d. In a preferred embodiment the apparatus and method are adapted to provide a distribution system for a cell therapy product, to extend the regulatory environment of the production site through transportation and processing at the point of care to the point of administration to the patient, allowing the product to be distributed within an unbroken chain of regulatory care. Data regarding regulatory compliance may be collected, analysed and reported automatically and an indication of compliance at the point of care may be generated automatically by the apparatus.

By handling is meant containing the biological product or subjecting it to a process as described herein above. By data tag is meant any component, subsystem or subassembly adapted to store data such that the data can be read without a wired connection. Preferably the data tag is adapted to be readable by RFID, though tags in the form of electronic subassemblies that communicate by radio, e.g. WiFi or Bluetooth are also envisaged. Wired connections to the device are within the scope of the invention but are not preferred.

Data regarding the device, biological material and parameters relating to processes through which the device and/or biological material has passed, such as process settings used by apparatus carrying out the process, or parameters measured from sensors forming part of the apparatus, is referred to herein as ‘device information’. Device information may be added to at each stage in the process, so forming a record that tracks the process stages through which the device and contents have passed.

In some embodiments device information may comprise data used by the apparatus to control one or more stages of the process, for example to provide instructions and settings to component apparatus to control their operation. The device information may comprise parameters specific to the biological material contained with the device to control its downstream process, for example at the point of care, and may in some embodiments be written onto a tag forming part of the device at the production site.

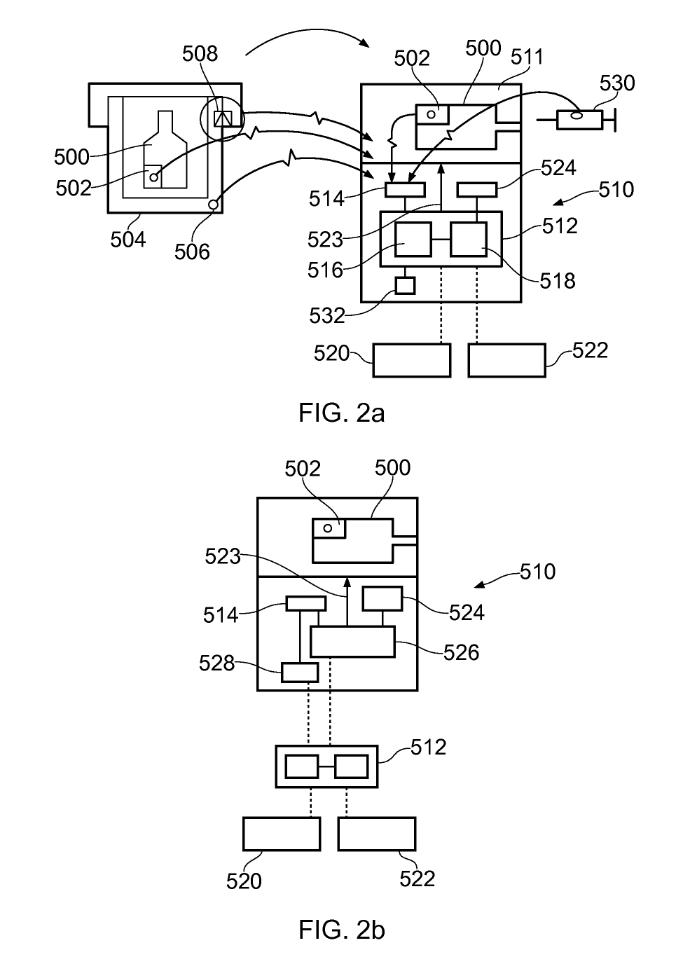

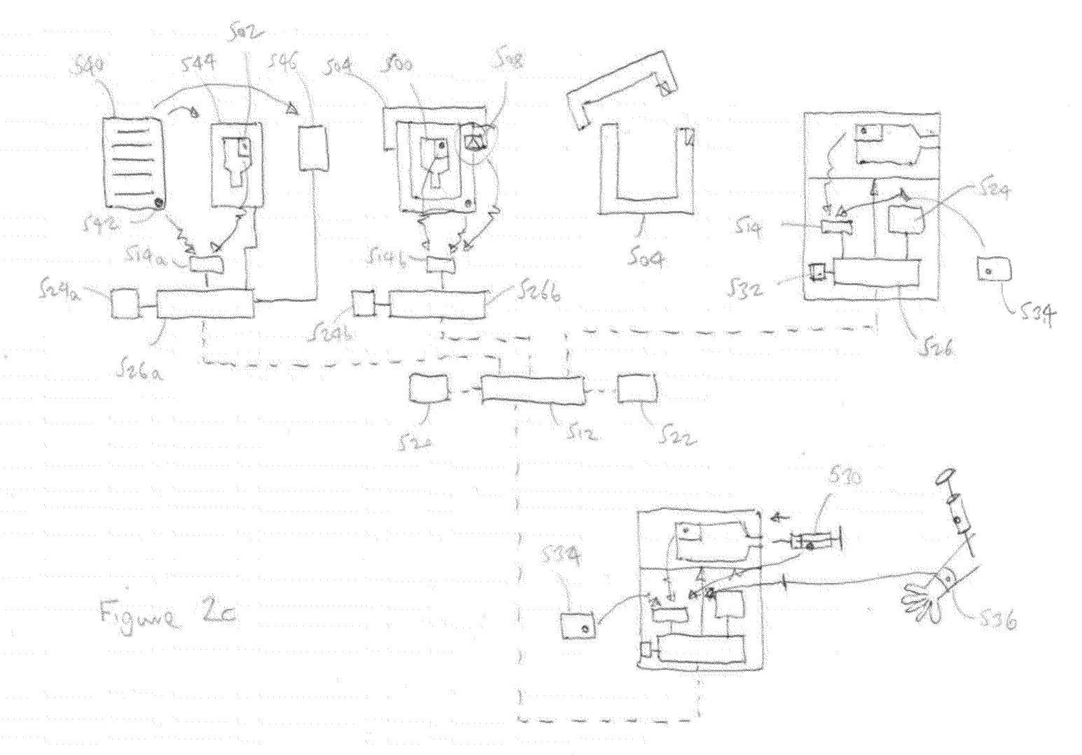

In an alternative embodiment the apparatus comprises one or more biological material handling devices comprising a read/write data tag and means to write data to the tag, wherein the data tag accepts, stores and maintains information regarding the material handling device during the processing of the biological material. Device information comprising device ID data, and other data regarding the biological material and processes, is thereby at least in part stored on the data tag and so is physically associated with the biological material handling device and so may travel with it during the stages of a multi-stage process. In one embodiment data may be exchanged between a first component apparatus, or device, and a second component apparatus by writing data to the tag at the time of process involving the first component apparatus and then reading data from the tag at the time of a process involving a second component apparatus. In an example application, data may be transferred by being written to the data tag by a first subsystem at a first location, the data tag then being transported along with the biological material handling device, the data being read by a second subsystem at a second location. Device information may therefore be recorded, stored and added to on the data tag, in a remote database or both. The data system and analysis means of the invention may use device information in one or both locations in monitoring regulatory compliance. Such a system may be understood with reference to FIGS. 2a-c and 3a-j in which the device information is written to and stored in part on the data tag on a device, and the data system may comprise a number of subsystems 514, 526 comprising a memory, and may have data linkages to HIS and LIMS, without the need for a central server 512.

In the embodiments herein where reference is made to a data tag being provided and data being read from the tag, it is understood that the tag may be a read/write tag and the process of data exchange between the tag and the data system may comprise both reading data from and writing data to the tag.

FIG. 2a shows an embodiment of an apparatus according to the invention comprising a cell product package 500 comprising a data tag 502 adapted to read and log temperature against time (referred to as a T/time log), a controlled temperature shipper 504 containing the cell product package and preferably comprising a data tag 506 containing data related to the shipper. The data tags may comprise an RFID read-only ID tag, an RFID read-only temperature sensor, an RFID T/time logging tag, or an electronic unit comprising one or more sensors, a memory and an RF communications unit. The shipper may optionally have sensor means 508 to detect opening of the lid of the shipper, for example a proximity sensor device and actuator pair plus subsystems as needed to allow reading by RFID. The apparatus further comprises a point of care processing device 510 having a location 511 at or within which the cell product package may be positioned and the process carried out by processing apparatus 510 on the cell product within package 500, preferably in a closed system manner. In this embodiment processing apparatus 510 comprises a data system 512 comprising a database 516 and analysis means in the form of a data program 518, and in communication with a terminal means 514 adapted to read the tag 502, and in preferred embodiments other RFID tags on further devices, as for example a patient administration device 530. In a preferred embodiment an output from the data system is used to control the process as shown by command pathway 523. Parameters from the process are logged by the data system, either as direct data inputs or by means of reading the T/time tag 502. The apparatus may be provided with an external control 532 to initiate the processing. Data linkages are shown as dotted lines and may be bi-directional and achieved by any means such as LAN, internet, wireless LAN or RF communications. Communication between the tag and the terminal may be read only or read/write, according to whether data is being stored on the tag or not.

The apparatus and method may be adapted to accommodate a cell product package 500 that comprises a bag to contain a cell product as known in the art, or is formed substantially as in any of the embodiments described herein. In some embodiments the package may comprise a rigid vial, and may comprise a tag assembly interoperable with the vial as described herein.

In use the data system 512 reads device information into the database from the tag 502, optionally from shipper 506 and lid opening sensor 508, optionally from an operator ID tag 534 (not shown) and from further tags associated with components of the administration process and with the patient (536). The package 500 is placed in the processing location 511 and processing is initiated. The data system receives data from the process and records one or more parameters in the database. The data program 518 runs in the data system, analyses device information from the tag and in the database and data from the process, and determines whether parameters from the point of care processing and previous process stages that the cell product has experienced were within compliance limits, and outputs data to compliance indicator means 524, and optionally the HIS 520 and/or LIMS 522. Device information stored on the tag or in the database may comprise the said target parameters and limits for the particular cell product in the package.

FIG. 2b shows an alternative embodiment in which the data system 512 is separate from the point of care processor, which now preferably has a control means 526 for controlling local operations and data acquisition while data association and analysis may be done in the remote data system 512. Data from the terminal 514 may pass to the data system via the control means 526 or separately via a communications unit 528.

FIG. 2c shows an embodiment of the invention comprising a data system 512 remote from the component apparatus, said component apparatus comprising a control means 526, 526a, 526b for local operation of the process and terminals 514, 514a, 514b that read/write data from/to the tag. Cell product is produced at a production site, shown schematically as in a cell factory 540, preferably comprising a data tag or other monitoring means 542 readable by a terminal 514a and control means 526a. At the production site, cell product is loaded into package 500 in a loading (i.e. a fill and finish) apparatus 544, preferably having a data linkage to the control means to upload data on the filling process. A QC process 546 is implemented to check the quality of the cell product and the fill and finish process, and data is passed to the data system, for example by means of the control means. The QC process then authorises release of the product, which may be done manually by an authorised person, and release data and other device data may be written into the database in the data system 512. The package 500 may then be placed in a controlled-environment shipper 504 ready for dispatch. The data system may read and record information from tags on the device, on the shipper and for example associated with an operator, and may be adapted to indicate clearance to dispatch. The data system may communicate with the LIMS and the HIS to notify that the cell product is in transit. Indicators 524, 524a, 524b may be provided to give information on compliance at one or more stages of the process and non-compliance may be used to halt the process or to initiate further stages of processing or QC.

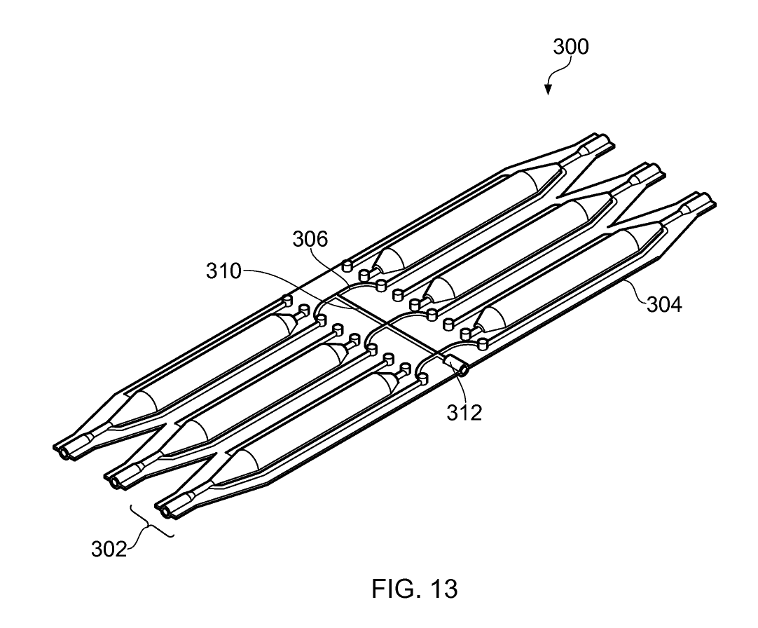

FIGS. 3a-j illustrate apparatus and a method according to the invention for the example of an autologous cell therapy process, with details of the stages of the process being illustrative only—it will be understood that the apparatus and method of the invention may be configured and used for a variety of different cell types, cell product formulations and processes, including allogeneic cell therapy processes, and that the method may comprise one or more of the steps shown and in some embodiments further steps. The apparatus and method may be applied to monitor and control a multi-stage process having processes carried out at more than one location: for example stages shown in FIGS. 13a-c may take place in a clinic, followed by transport to a production facility (e.g. a CMO), where stages in FIGS. 13d-h take place, then transport to a clinic and processing at the point of care (FIGS. 13i, j), illustrated here by a process of warming the cell product prior to administration, but which may involve further process steps in alternative embodiments.

FIG. 3a shows a first step in a method according to the invention comprising preparation for a biopsy procedure, in which tags associated with one or more of the patient 536, a biopsy device 550 and tag 552, a biopsy vial 554 and tag 556, patient/protocol documentation 560 and tag 562, clinician ID 534 are read by terminal means 514a, the data forming part of the device information for the biopsy vial, and being captured by control means 526a and communicated to a server 512 forming part of the data system. In a further step in FIG. 3b a biopsy 564 is taken from a patient and placed in the biopsy vial. The process is tracked and monitored by the data system by means of reading of data tags by the terminal means 526b. Terminal 514b and control means 526b used in this stage of the process may be distinct from terminal means 514a and control means 526a used earlier or may be the same, according to the location of the processes. Device information may be analysed by a data program operating in either the control means 526b or the server 512 to determine regulatory compliance of the biopsy process. For example, a complete, correct and timely sequence of operations may be monitored by the timing and sequence of reading of the tags. Indicator 524b may indicate compliance at this stage. Multiple terminal means 514 may be provided at a given location to allow more precise monitoring, for example in different parts of the process location. In a preferred embodiment in which the device tag 556 is a read/write tag, device information may be written to the tag allowing it to be read in later process stages. Data determining compliance at a given stage may be part of the device information written to the tag and/or stored in the database.

In a further step in FIG. 3c the loaded biopsy vial is placed in a controlled environment shipper 504, which may be provided with a T/time tag 506 positioned to read the temperature of the vial. Alternatively in a preferred embodiment the device tag 556 is a T/time tag. Tags, optionally including a lid open/closed sensor 508, are read by the terminal means 514c and data is added to the device information. Compliance with the process protocol may be checked, indicated and flagged in the database and/or device tag 552 as before. The biopsy is then shipped to the production location (e.g. a CMO). In a further step as shown in FIG. 3d tags are read on arrival at the CMO and device information may be communicated to the database by means of a data linkage shown as 572. In a further step cells may be isolated from the biopsy in a process monitored by the data system as shown in FIG. 3e. Tags associated with the biopsy vial 554, one or more process consumables such as T-flasks 564 and media 566, and process documentation 568 such as a batch follower, and operator ID 570 may be read and associated with device information in the database.

FIG. 3f shows a further step in which cells of interest are expanded by means of passage from a first T-flask 564 to further T-flasks 574. Tags associated with T flask 564 and T-flasks 574, and media 576 and optionally other consumables are read by terminal means 514f and data passed to the data system comprising control means 526f and remote server 512. Optionally the step may comprise a further transfer from T-flasks 574 to a larger scale cell expansion means such as cell factory 580, preferably also having data acquisition means and means to communicate data to the data system. The data acquisition means is illustrated as a data tag 582, which may for example be an ID tag and/or a T/time tag, but other data acquisition means such as sensors or a control subsystem associated with the cell expansion means are included within scope, which may be connected by LAN or RF data links. Following expansion a cell product may be formulated and loaded into a transport vial 584, preferably comprising a tag 586 that may be a T/time tag, and which may be a tag assembly as disclosed herein. A portion of the product may be sent for QC, optionally in a further tagged vial 588. The QC process is represented by unit 546, which is preferably in data communication with the data system for example via data linkage to control means 526g. Indicator means 524g may indicate release to dispatch the product.

FIG. 3h shows the cell product packed in a controlled environment shipper 504, Documentation 585 may also be packed alongside the product and comprise a further tag. The data system preferably checks that all components of the shipper are packed, and may check that the lid is closed by means of proximity sensors provided on the lid and body of the shipping container as shown by 508. Indicator means 524h is preferably provided to indicate a release to ship the product. The product vial 584 may comprise a tag, preferably a T/time tag. In an alternative embodiment, the vial may contain an ID tag, and may be used as part of a tag assembly 600 comprising a T/time tag 602. The tag assembly is preferably an embodiment as disclosed herein.

The product may now be transported to the clinic. FIG. 3i illustrates the method on arrival at the clinic, where the data system may read the tags by means of a further terminal means and control means, preferably reading T/time data from the tag associated with the product vial (586) or tag assembly (602), and optionally data from other sensors where provided such as lid open/closed sensor 508. The data system preferably then stores the shipping data in the database, and may indicate compliance or give warning by indicator means 524i.

This embodiment describes a simple example of a point-of-use processing steo in which the product is warmed from a first temperature suitable for shipping/storage to a second temperature suitable for administration to a patient. For example, the product may be transported and stored cool, such as for example at 2-8C, and then be warmed before administration. In alternative embodiments the product may be transported and stored frozen, in which case the warming means may be adapted to thaw the product. The illustration in FIG. 3i shows a process of warming in-situ within the shipper, though other point-of-use processing apparatus, steps and operations are within scope of the invention, for example point of use processing involving centrifugation, filtration and flow control for washing and re-suspension of a cell product as described with reference to cell product packages as described herein, and the features and operations described below may also be included in apparatus for those processes. In the embodiment in FIG. 3i the shipper comprises means 590 for warming the cell product, for example to thaw the cell product or to warm the cell product from a first temperature above zero for chilled shipping to a second temperature suitable for administration of the cell product to a patient. Warming means 590 may comprise an actively heated temperature control means forming part of a controlled temperature shipper as disclosed in pending application WO06/097751, further comprising a warming control 594, for example a manual control. Warming means 590 may comprise a data tag or RF communication means 592, which may be adapted to receive signals from the terminal means 514h. In one embodiment the data system optionally enables actuation of the warming control means 594 by means of signals received by the warming means. When the product is needed for administration, the warming process is initiated by the warming control means 594, and readiness for administration may be indicated by an indicator means 524i or by an indicator provided as part of the warming means 590.

FIG. 3j shows the process of administration to the patient. The shipping container is opened, the product vial is removed and product is loaded into an administration device 604 preferably comprising a data tag 606. In preferred embodiments T/time tracking of the product vial is continued during this process, for example by means of a T/time tag 586 or by means of a tag assembly 600 in which the vial may be housed. Preferably the tag assembly has a defined thermal mass, and may have specific features to control its thermal behaviour, in order to control the temperature of the product in the product vial. Such embodiments are disclosed herein. The product may then be administered to the patient. The data system may read one or more of tags associated with the shipper, the product vial, the tag assembly where present, the admin device, patient ID from a patient tag 608, clinician ID from an ID means 534, and optionally other required equipment, conditions or personnel, and may record the process of treatment in a database and may exchange data with one or both of the HIS and LIMS.

The apparatus may indicate compliance with regulatory requirements by means of an indicator means 524j and may give a ‘go/no go’ indication to clinicians based on one or more of: data in the database, which may include one or more of: device information associated with the biological material containing device, e.g. the product vial in this example, data regarding the processes that the device has experienced; data recorded from sensors at one or more stages of the process, such as in this example T/time data from the data tags associated with the biopsy and product vials; time data indicating that the processes were completed within such time limits as may be set out regarding the shelf life of the product; tamper evidence, for example opening of the shipping device lid by non-authorised personnel.

In FIG. 3j the administration device is shown as a syringe, and loading of the product simply as drawing the product up into the syringe from the vial. In alternative embodiments the administration device may be for example a catheter or a bag for infusion. In an embodiment, the administration device is included as part of a sterile package comprising the product vial.

The invention provides means to monitor the conditions of a biological material being handled between discrete processes, so as to allow a continuous or semi-continuous monitoring of the biological material from a starting point in the process to an end point, with the objective of providing data for analysis to determine compliance with a pre-determined set of conditions, for example to allow an indication of regulatory compliance at the end of the process, such as for a cell therapy product an indication at the point of care.

Accordingly the invention provides a biological material handling device comprising means to monitor, to report and in embodiments to control the conditions experienced by the biological product, for example as referred to above as a vial assembly, the terms ‘vial assembly’ and ‘biological material handling device’ having identical meanings herein.

In a further aspect the invention provides a transport apparatus for transporting a biological material comprising a vial assembly, the vial assembly comprising: A sealable vial or other container adapted to contain the biological material in a liquid-tight manner—this container will be referred to as a vial for simplicity, but may comprise a jar, plate, tube or other form as known in the art. The vial may be a standard product as known in the art or may be a specific proprietary design;

A tag assembly interoperable with the vial that attaches to the vial, the tag assembly comprising one or more data tags.

The tag assembly may attach to the vial by any means as known in the art, for example by push fit, snap fit, screw fit, welding, ultrasonic or adhesive bonding.

The tag assembly may comprise a component adapted to interact with one or more features on the vial to attach to the vial, for example to snap into place.

The tag assembly may attach such that it is readily detachable, detachable manually with special procedures, detachable with a tool, or not detachable without damage to one or both of the vial and the tag assembly.

The tag assembly may be permanently attached or bonded to the vial, or formed as part of the vial in a common fabrication process.

The tag assembly may be re-usable, for example reversibly attachable to the vial, so that once the tag assembly has been used with a housing and data has been transferred to/from the tag assembly, the vial may be discarded and the tag assembly used with a further vial.

The tag assembly may comprise sensors that record conditions in the vicinity of the tag assembly. The tag assembly may be adapted to record one or more conditions in the vicinity of the vial, for example, temperature, events such as sealing and opening of the vial, shock, humidity, light intensity, X-ray intensity, orientation, gas concentration.

The tag assembly may be adapted such that the vial locates adjacent to one or more sensors provided as part of the tag assembly. For example, a tag assembly may have a location for a vial such that it is in good thermal contact with a temperature sensing location, allowing a temperature sensor associated with the tag assembly to read a representative temperature for the vial and its contents.

The tag assembly may comprise a closable housing enclosing the vial, the housing having a lid, the lid controlling access to the vial by an operator. The housing may comprise a liquid-tight seal and may act as a secondary containment in case of leakage of the vial.

The housing may be adapted provide compliance with shipping regulations, for example UN3373 for biosamples and blood products.

The lid may have features such as tamper-evidence or one or more sensors to detect when the lid is closed or opened. Such a sensor may communicate with a tag forming part of the tag assembly to log opening and closing and may comprise a proximity sensor, for example active through magnetism or actuation force

The housing may define a sealed space in fluid communication with the outside of the vial, that space being capable of holding an atmosphere different from that outside the housing. The space may for example be adapted to contain a chosen gas atmosphere, such as with known CO2/O2 content, humidity, or pressure. The housing may be adapted to receive a gas line to establish such an atmosphere, and may have more or more gas ports to facilitate this. The gas port(s) may be sealable, either reversibly or permanently, once the chosen gas atmosphere is present. The vial may be chosen or adapted to allow gas exchange between the space and the contents of the vial.

The vial may be a gas/media vial as disclosed in pending application WO08/129,300.

One or more tags on the tag assembly may be adapted to interact with further tags external to the tag assembly, for example as may be worn by an operator who uses the transport apparatus, so that information such as the identity of an operator, time of use, time of opening/closing of the lid, may be recorded. One or more tags may communicate with a remote data system, logging an association between an opening/closing event and proximity to an operators' tag in the remote data system.

The tag assembly may be designed to have common design for the tags, sensors and other electronics where necessary, while using a specific form to interoperate with a chosen form of vial. In this way a wide variety of vials may be useable in the invention without costly variants in the manufacturing processes.

Such a transport apparatus has advantages in production and use which may include: use of existing, standard vials; ability to cater for a variety of standard vials with a common tag assembly product; use of standard RFID interfacing to read from/write to the tags.

Such a transport apparatus has advantages in use including: easy snap in place of a vial into the tag assembly; automatic tracking of the use of the vial, temperature or other conditions; re-usability of the tag assembly with further vials, or, according to the usage pattern, disposal of the vial plus tag assembly after a single use.

The transport apparatus may further comprise a controlled environment transport container or ‘shipper’ adapted to contain the vial assembly. The shipper may comprise a location in which the vial assembly locates, in order to provide control of its environment. The location may comprise a lid and may provide a sealed environment in the event of a leak from the vial assembly. The location may be adapted to be fluid-tight and to allow containment of a chosen gas atmosphere, and/or gas pressure, within it.

The shipper may be of a form as known in the art to provide temperature controlled within a range. The shipper may use an active temperature control principle, for example as disclosed in pending application WO06/097751. The location may be shaped to receive the vial assembly snugly, to give optimum control over its temperature.

The shipper may comprise a data tag holding data that may be associated with data from the tag assembly, in storage in the tag assembly or in a remote data system, so recording use of the shipper with the vial assembly. The shipper may comprise its own logging system for data such as temperature, which vial assemblies have been used with it, or other variables.

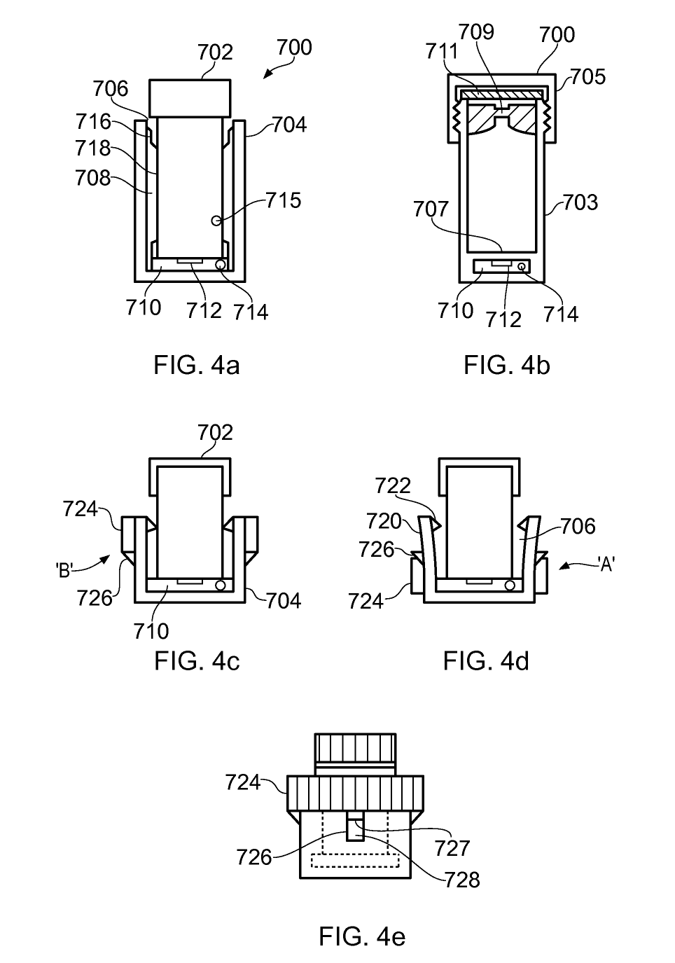

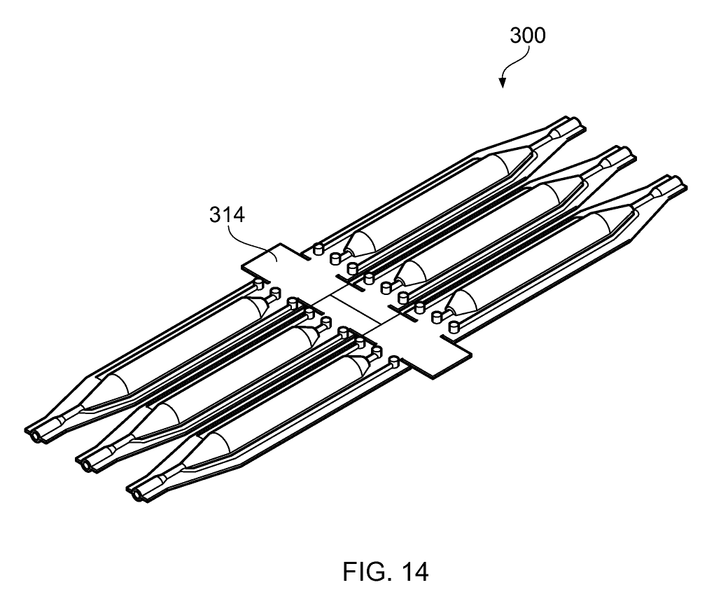

FIG. 4a shows an embodiment of a biological material handling device 700 according to the invention, comprising a vial 702 and a tag assembly 704 adapted to be interoperable with the vial, into which the vial may be inserted, attached or bonded, either permanently at the point of manufacture or later (i.e. on first use), or in alternative embodiments removably, to allow for re-use of the tag assembly with a new vial. The tag assembly comprises a recess or location 706 for the vial and means to retain the vial within the tag assembly, either permanently or reversibly. In the embodiment in FIG. 14a this means comprises one or more features 708 adapted to have an interference fit along a portion of the body of the vial, so locating it firmly. In a preferred embodiment the features comprise splines adapted to engage with the surface of the vial so as to grip it tightly. The vial may be a standard product with a rigid surface and the splines may be sharp so as to engage closely with or dig into the surface and so to hold the vial. In this case the splines preferably have lead-in portions 716 and sharp portions 718 that engage the surface. In an alternative embodiment the vial may have features formed on or as part of it that in use engage with clip means provided on the recess 706, so acting to hold the vial in place.

The tag assembly comprises a temperature monitoring means 710 comprising a temperature sensor 712 and a communication means 714, for example a RFID communications means. These features may be provided in a T/time RFID tag as known in the art, mounted within the tag assembly. The vial preferably mounts with the sensor in contact with the vial, so monitoring a temperature indicative of the temperature of the biological material within the vial. The vial itself may also comprise an ID tag 715. In an alternatively embodiment the sensor(s), communication means and memory may form part of an electronic subassembly and be distributed in one or more different locations as part of the device. The tag or subassembly may comprise a battery or RF-energised power source as known in the art. The formation of the device from a container and a separately-formed tag assembly has advantages in practice, including: the container or vial may be manufactured separately allowing low-cost, standard manufacturing as presently done without changes to the process to allow for incorporation of electronic components; sterilisation of the container may be done without passing the electronic components through the sterilisation process, which may limit the type of sterilisation and components that are usable; cost optimisation of each component separately. In some embodiments the container or vial may comprise a substantially rigid sealable containment, such as a screw-top vial or septum-sealed vial; others may comprise a flexible containment such as a bag, the tag assembly being adapted to hold the bag in such a way as to present one or more sensors in the vicinity of the bag, preferably in contact with it, so as to monitor conditions of, and hence within, the bag.

FIG. 4b shows an alternative embodiment of a device according to the invention which comprises a moulded plastic housing 703 defining a product space within it, and having within a thickness of its walls, preferably in the base, a temperature monitoring means 710 comprising a temperature sensor 712 and a communication means 714 as described before, preferably also comprising a power source such as an RF-driven power source or a battery. Preferably the wall region 707 between the temperature sensor 712 and the product space is thin to give temperature readings representative of the temperature of the contents. The device may further comprise a septum means 709 and tear-off seal 711 as known in the art.

FIGS. 4c-4e show an embodiment of the device comprising a tag assembly adapted for easy manual insertion into, and optionally release of the vial from, the tag assembly. The tag assembly in this embodiment comprises one or more regions 720 that are flexible and may be deformed towards the vial in order to bring features 722 to bear on the wall of the vial. Such features are preferably adapted to engage and to engage with or dig into the exterior of the vial, so retaining it, and regions 720 are preferably formed so that when relaxed they are sprung apart as shown in FIG. 14d to allow easy entry of the vial into the recess 706 past the features 722. A feature adapted to compress the regions 720 is provided, in this embodiment the compression ring 724, which is adapted to move vertically from an open position A in FIG. 14d to a closed position B in FIG. 14c, so as to cause features 722 to move inwards to bear on the exterior of the vial. Locking features 726 may be provided to hold compression ring 724 in position B, these being for example in the form of a ratchet tooth 727 on a sprung flexible arm 728 formed as part of the housing of the tag assembly as shown in FIG. 14e. In this way a low-force manual action may translate to a high gripping force applied by the features 722.

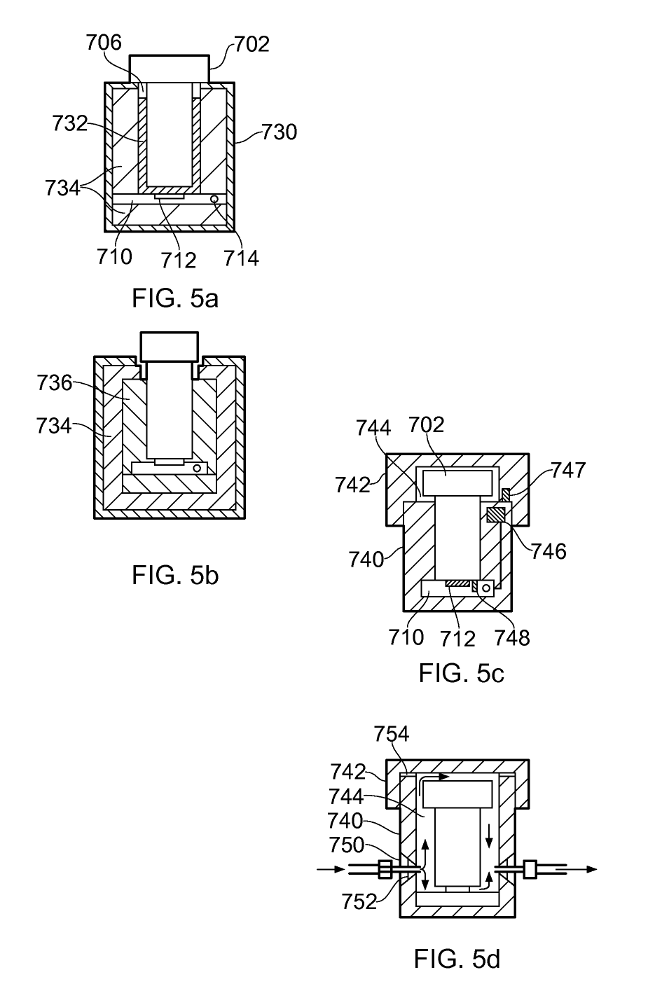

FIG. 5a shows a biological material container according to the invention in which the tag assembly further comprises insulation means 734, for example a layer of insulating material or an air gap between the vial and the wall of the recess 706, which in use may function to reduce sudden changes in temperature of the vial during movement from one controlled temperature environment to another. In a further embodiment a layer of conductive material 732 may surround the vial and may be in contact with it, the layer being in contact with the temperature sensor 712 so as to increase the ability of the sensor 712 to register a temperature characteristic of the vial as a whole, and hence the biological material, rather than that of a localised area of contact.

FIG. 5b shows a further embodiment in which the tag assembly is adapted to have a greater thermal mass that that of the vial so helping resist sudden changes of temperature. A component material 736 may be chosen to have an appropriate volume and specific heat capacity, and in a preferred embodiment may comprise a phase change material adapted to buffer the temperature of the interior of the tag assembly to a chosen range.

FIG. 5c shows a further embodiment in which the tag assembly comprises a housing 740 and lid 742 that together define a closable space 744 surrounding the vial 702 when in place. The lid acts to control and in some embodiments to monitor access to the vial. The tag assembly preferably comprises means to detect when the lid is opened and/or the vial is removed, for example a proximity sensor 746 and associated actuator 747 may signal to the electronic subsystem 710 that the lid has been opened; alternatively the subsystem 710 may comprise a sensor 748 that detects the presence of the vial 702, for example by means of a proximity sensor and associated actuator provided as part of the vial, or a pressure switch actuated by pressure from the vial being pressed down when the lid is closed and in a preferred embodiment then released when the lid is opened—spring means may be provided to hold the vial in place once the lid is closed, and may also cause suitable movement of the vial when the lid is opened and closed. Further sensor means, such as capacitive proximity sensing, may be provided to detect the presence of the vial. Opening and closing events may be logged by the electronic subsystem 710 for later reading and analysis by a data system in communication with the electronic subsystem.

FIG. 5d shows a further embodiment in which the biological material container further comprises a lid 742 that forms a substantially gas-tight seal to the housing 740 and further comprises means to fill space 744 with a chosen gas atmosphere. In a preferred embodiment the filling means comprises a gas inlet port 750 comprising a closure 752, for example a septum, and preferably an outlet and further closure to allow flushing of the space 744 with the chosen gas. The gas may be chosen according to the biological material and in use may act to reduce diffusion of gas such as oxygen and carbon dioxide through the wall and lid of the vial 702, so helping to reduce changes in the gas concentration and pH within the vial.

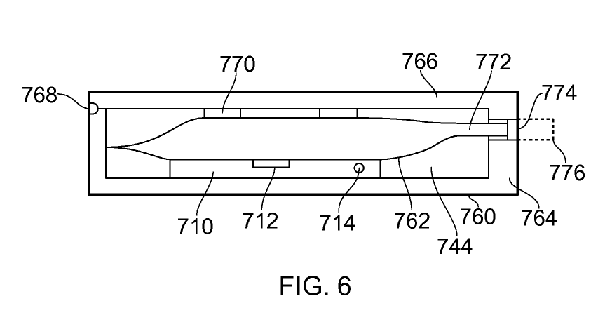

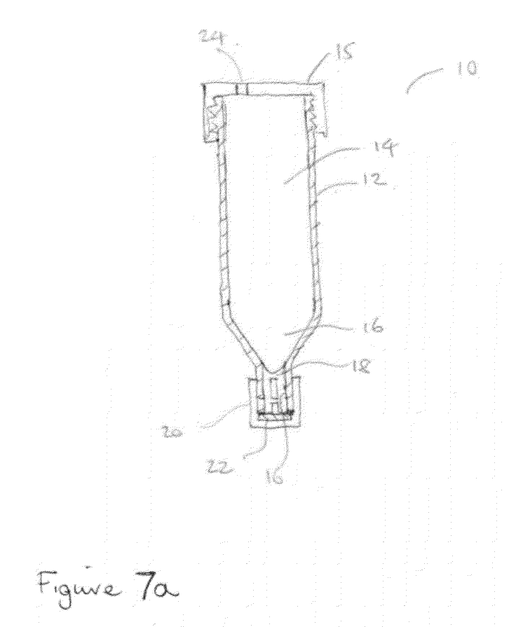

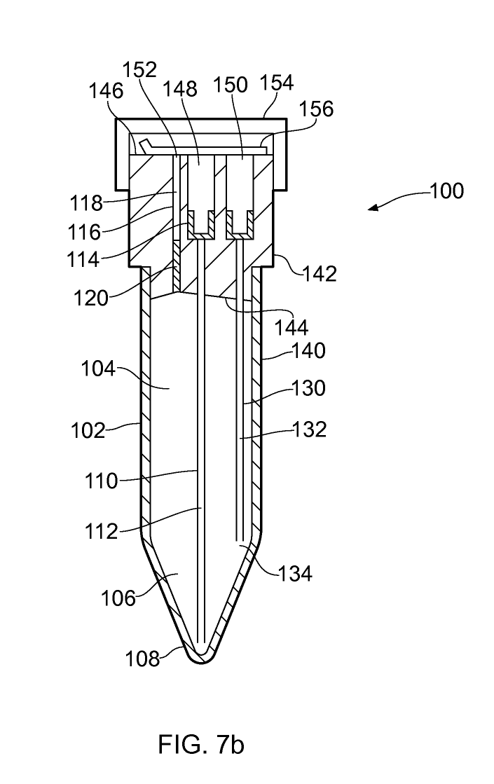

FIG. 6 shows a further embodiment adapted to retain and monitor a biological product stored in a bag format. The biological material container comprises a housing 760 adapted to hold bag 762 and further comprises an electronic subsystem or T/time tag 710, comprising a temperature sensor 712 and communication means 714. The housing preferably comprises a base 764 having a recess to accommodate the bag and a lid 766, optionally hinged to base 764 by hinge 768, that closes on base 764 to retain the bag and preferably to hold the bag against the temperature sensor 712. Features or compliant components 770 may be provided to assist this. The bag preferably has an outlet tube 772, that may extend beyond the housing as at 776; alternatively the housing may be closed by a closure 774, and the housing may optionally be substantially gas-tight so as to retain a chosen gas atmosphere in the space 744 within the housing; preferably means are provided to flush this space with gas such as are shown in FIG. 5d for example. Closure 744 may be adapted in some embodiments to be pierceable or removable to allow access to the bag outlet tube 772.

In a further aspect the invention provides a cell product package adapted for use of centrifugation and suited for use in an apparatus for handling a biological material such as a cell product as described above.

According to a first embodiment, referring to FIG. 7a, the invention provides a cell product package (10) adapted for centrifugation, comprising a housing (12) that defines a product space (14), the product space having an elongated form and a tapering profile to define a tapered region (16) at the distal end, and a closure (15) at the proximal end, further comprising a first fluid conduit (16) defining a fluid pathway that opens to the interior of the product space at or adjacent to the distal end, the first fluid pathway being closed by a closure (18) such as a septum means, and adapted to allow withdrawal of fluid along the pathway by an external flow means in fluid connection with the pathway.

Referring to FIG. 7b, in a further embodiment the invention provides a cell product package (100) adapted for use in centrifugation comprising:

A housing (102) that defines a product space (104) adapted to contain a cell product, the product space having an elongated form with a long axis and a tapering profile defining a taper region (106) within the distal end of the product space, the housing being adapted for use in centrifugation;

A first fluid conduit (110) defining a fluid pathway that opens to the interior of the product space at or adjacent to the distal end;

A closure device (114) closing the first fluid conduit, the closure device being openable to allow fluid to flow along the first fluid conduit;

A second fluid conduit (116) defining a second fluid pathway that opens to the interior of the product space at or adjacent to the proximal end;

Breather means (120) in fluid communication with the second fluid pathway, adapted to allow equalisation of pressure in the product space when fluid is flowed along the first fluid pathway.

The cell product package may serve in use to facilitate one or more of: concentration of cells in a cell product from a first concentration suitable for transport and/or culture of the cells to a second concentration suitable for administration to a patient; washing of the cell product, i.e. concentration followed by re-suspension of cells in a second liquid suitable for administration to a patient, in which a first medium suitable for transport and/or culture of the cells is partially or substantially replaced with the second liquid; washing as above, followed by concentration of the cells to form a concentrated cell product, at a second concentration suitable for administration to a patient.

The housing may be of rigid, semi-rigid or flexible form.

The product space may be of any appropriate cross sectional shape, for example substantially one of: circular, semi-circular, trapezoid or rectangular.

The product space may be of dimensions appropriate to the volume of cell product to be contained. The volume of the product space may be for example in the range 1000 to 100 ml, 100 to 10 ml, 10 to 1 ml or 1 to 0.1 ml. The housing may be narrower in cross section than it is long, with the aspect ratio of length to typical dimension perpendicular to the axis of the housing for example in the range 1:1 to 20:1.

The housing may have a length along its axis in the range of 20 to 10 cm, 10 to 5 cm, or less than 5 cm and a dimension perpendicular to the axis in the range of 5 to 3 cm, 3 to 1 cm or less than 1 cm.

The housing may be adapted for centrifugation by comprising features such as: appropriate material; strength and fabrication method appropriate for the stresses encountered in centrifugation; axial symmetry in shape or distribution of mass to promote uniform behaviour during centrifugation; dimensions suitable to fit within a standard commercially-available centrifuge.

The taper may be uniform, for example conical or semi-conical, or non-uniform, for example with a cross-sectional dimension decreasing in one or more steps. The taper may comprise a change in cross-sectional shape, for example from trapezoidal to circular or semi-circular.

The taper is adapted to facilitate concentration of cells from a cell product comprising a suspension of cells towards the distal end of the product space, leaving a supernatant liquid above the concentration zone. The angle, or step size(s) of the taper are adapted to facilitate concentration.

The first fluid conduit may be adapted to allow flow of a cell product along the first fluid pathway for example to: load a cell product into the product space; to remove a cell product from the product space; to flow a liquid medium into the product space; to withdraw a liquid medium from the product space; to withdraw a cell product from the product space, for example into a patient administration device such as a syringe, syringe needle or catheter.

The first fluid conduit may extend within the housing substantially parallel to the axis of the housing and may have a first opening adjacent to the narrow end of the taper and a second opening at the proximal end of the housing. The first fluid conduit may be substantially coaxial with the product space or may lie away from the axis, for example adjacent to a wall of the product space.

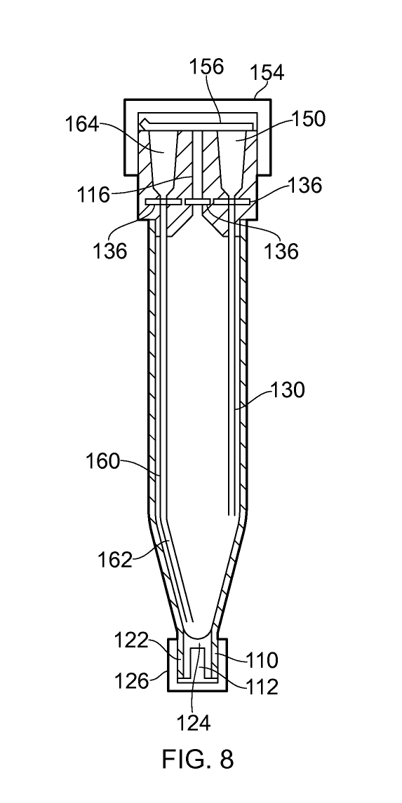

Referring to FIG. 8, the first fluid conduit may be formed within an extension (122) to the housing beyond the taper and may be coaxial with the product space. The first fluid pathway may open to the product space at, or adjacent to, the distal end of the taper. This arrangement may facilitate removal of cell product along the first fluid pathway towards a patient administration device such as a syringe needle.

The first fluid conduit may be closed by a closure device (124) such as a septum or valve that may be pierced or opened by a tool such as a tube, projecting plug portion, pipette tip, syringe, needle or other form of fluid connection means. The closure device may be adapted to re-seal after opening. The closure means may be a septum that may be pierced by a patient administration device such as a syringe needle or catheter in order to withdraw cell product from the tapered section of the product space.

The closure device may be covered by an outer sterile closure (126) such as a snap-off cap or tear-off label, so ensuring sterility of the closure device outer surface until access to the cell product is needed.

The first fluid pathway may be adapted to supply liquid medium to the product space, in particular to supply liquid in the vicinity of the tapered portion.

The second fluid conduit may be adapted to allow flow of fluid into the product space to equalise wholly or partially changes of pressure in the product space when a cell product is withdrawn or introduced via the first fluid conduit.

The second fluid pathway may be closed by a breather means such as a porous membrane or a valve in order to allow air to flow along the second fluid pathway when a cell product is withdrawn or introduced via the first fluid conduit.

The second fluid conduit may be adapted for use in filling the product space with cell product, air from the product space being expelled via the first fluid pathway. The first fluid pathway may be provided with a breather means as above to facilitate this.

Referring to FIGS. 7b and 8, the cell product package may comprise a third fluid conduit (130) forming a third fluid pathway extending from a point within the product space to a point at or adjacent to the proximal end of the housing. The third fluid conduit may open at a region (134) in the product housing adjacent to the taper, for example at a region within the taper adjacent to the level at which the boundary between the concentrated cell product and the supernatant liquid will form following centrifugation. Supernatant liquid may be withdrawn along the third fluid conduit leaving concentrated cell product in situ in the taper region.

The third fluid conduit may extend within the housing substantially parallel to the axis, and may be coaxial or nearly so with the axis. It may be located away from the axis, for example adjacent to a wall of the product space.

Referring to FIG. 8, the cell product package may comprise a fourth fluid conduit (160) forming a fourth fluid pathway extending from a point adjacent to the distal end of the product space or from a junction with the first fluid pathway adjacent to the distal end of the product space, leading via the fourth fluid conduit to a further port (164). Such a fourth fluid pathway may be used for example to introduce fluid into a region adjacent to the distal end of the product space without that fluid flowing along the first fluid pathway, which may then be reserved for flow of cellular product.

Dimensions of the first fluid conduit may be chosen according to whether liquid medium, cell product or concentrated cell product are to pass along it. For example, a cross-sectional dimension of a conduit may be in the range less than 0.1 mm, 0.1 to 0.5 mm, 0.5 to 5 mm.

The fluid conduits may be formed in a number of ways known in the art. They may comprise tubular structures formed or mounted within the housing. For example tubular sections extending within the product space to open at the appropriate region. The tubes may be formed from plastic tubing sections, metal section such as syringe tubing and may be rigid or flexible. The tubes may be formed as part of one or more moulded components mounted on or formed as part of the housing. More than one fluid conduit may be formed as part of a component, for example a dual lumen tube or moulding.

One or more of the fluid pathways may comprise a filter, such as a 0.22 um pore filter, to prevent contamination of the interior of the device. The filter(s) may be provided as one or more substantially planar component(s) through which the fluid pathway passes; the planar component(s) may be located within the housing at the proximal end.

The housing may be formed for example by moulding and may comprise a single component or more than one. The housing may comprise a tubular component having a taper at the distal end and a cap or closure component that seals to the tubular component to form a closed product space. One or more fluid conduits may extend from the cap component into the tubular component. One or more fluid conduits may be formed as part of the tubular component or attached to it. One or more fluid conduits may be formed partially within the cap component, and the fluid pathways may pass through the cap component from a first, inner face in contact with the product space to a second outer face. One or more filter component(s) may be provided within or mounted on the cap component to introduce a filter element into the fluid pathway(s).

The housing may be substantially circular in cross-section, for example in the form of a moulded tube. The housing may be substantially rectangular, and may be assembled from a layered assembly of substantially planar components with recesses or through channels within them defining walls of the product space, conduits and reservoirs where present. For example, the housing may be formed from a body part comprising recesses and/or channels with one or more lid components mounted on one or both faces. The housing may be formed from a layered assembly comprising a profiled first component comprising recesses and/or channels bonded to a substantially planar component to close the recesses and/or channels.

One or more fluid pathways may extend to openings or ports in the cap, adapted to connect to further fluid components, for example, fluid connections, syringes, needles. Ports may be for example adapted to accept standard fluid connections such as Luer fittings. Ports may be closed by valves, such as flap valves or septum valves comprising a slit, as known in the art. One or more fluid pathways, for example the second fluid pathway, may extend to a breather port opening to the second surface of the cap.

The ports and/or the cap may be closed by a sterile closure such as a snap-off cap (154) or a tear-off strip (156) to maintain sterility of the ports and the fluid pathway until the time of use.

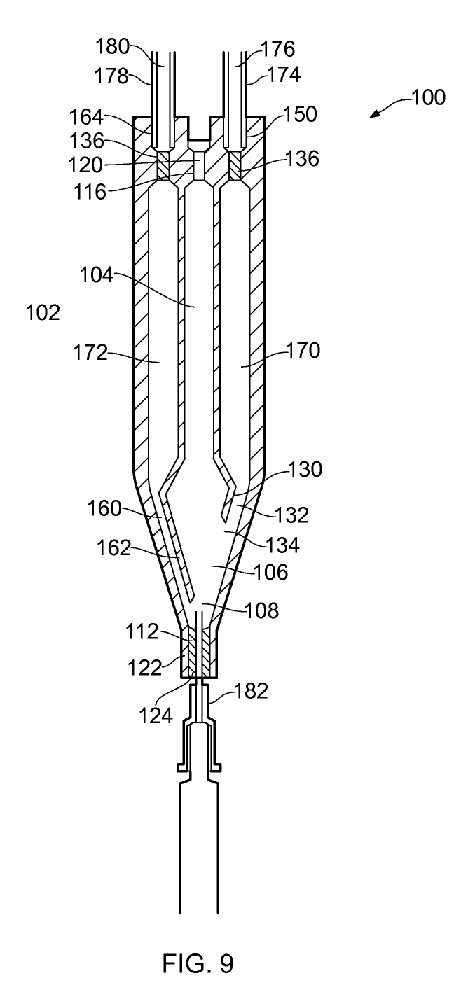

Referring to FIG. 9, the cell product package may further comprise one or more reservoirs (170, 172) that may form part of one or more fluid pathways. Such reservoirs may comprise reservoirs (172) for cell culture medium, saline or other medium suitable for suspension of cell product for administration to patients, and wash or waste reservoirs (170).

A reservoir (172) for saline solution may be provided as part of the first fluid pathway.

A reservoir for waste solutions (170) may be provided as part of the second or third fluid pathway.

Further reservoirs for further fluids, for example saline or cell culture medium, may be provided as part of further fluid pathways additional to the first, second or third.

One or more reservoirs may be filled at the point of filling cell product into the product space, for example the saline reservoir, and the cell product package sealed and transported containing the saline. The waste reservoir may be empty following the point of filling or may be partially filled.

The device may be adapted so that once filled, the only liquid that is removed from the device is processed cell product: other liquids are retained within it. Actuation of flow of liquids within the device may be by gas pressure at the ports leading to the fluid pathways.

The device may operate as a closed system, with no open interfaces across which liquids will travel, until the point at which concentrated cell product is withdrawn from the device.

The cell product package may comprise a layered assembly comprising two or more substantially planar components. The layered components may be rigid, flexible or only partially flexible, and may comprise material and use fabrication techniques used in laminating technology as known in the art for assembly of fluidic components. The layered assembly might comprise a product space having an elongated form as before, with a taper at the distal end. The product space may be defined as a raised profile in a substantially planer rigid component layer. A flexible component may enclose or be bonded to one or more rigid components so as to provide a layered assembly that has flexible regions and less flexible, or rigid, regions. The layered assembly may comprise one layer of flexible material bonded to a rigid component, or more than one layer of flexible material having as least two layers bonded together to enclose a rigid component. The product space may be defined by a rigid or semi-rigid spacer component bonded between two layers of flexible material, defining a rigid space within the layered assembly with an enlarged profile on a first face of the layered assembly. The spacer component may have a larger cross sectional dimension at a proximal end and a smaller at the distal end, so producing a tapered space when the components are bonded together. The spacer component may be adapted to produce for example a semi-circular, polygonal or trapezoidal cross sectional shape. Dimensions of the product space may be as before.

The cell product packages may comprise a data tag, for example an RFID data tag, which might be included in the package during the assembly process, for example embedded in the package and sealed in place under one of the flexible layers.

The product package may comprise one or more fluid conduits defining fluid pathways as before. The one or more fluid conduits may be defined as raised regions in a substantially rigid and planar layer component bonded to a substantially planar sheet component. They may be defined as passages between layers of flexible material, or may comprise tubular or non-tubular forming elements to facilitate definition of the conduits. The conduits may have the same functions as described above. One or more conduits may be comprise insert components that may define or retain for example a septum, a valve, such as a septum comprising a preformed slit; a breather component, a filter or other component in a fluid pathway.

Conduits may have a raised or enlarged profile on one side of the layered assembly and a flatter profile on the other. The layered assembly may comprise one, two, three or more layers of flexible material bonded together. Conduits may be formed with their enlarged profile on the first face of the layered assembly in common with the product space or the other, second face. For example, in a three layer layered assembly comprising a product space formed between layers 1 and 2, conduits may be formed between layers 1 and 2 or between layers 2 and 3, or both. Vias may be provided linking conduits on the first face and on the second face. Conduits on the first face may cross conduits on the second face without joining.

A first fluid pathway may be formed as a first conduit between two layers of bonded material, the conduit extending from the distal end of the product space, and terminating in a component comprising a septum adapted or withdrawal of cell product following centrifugation. The septum might be sealed with a sterile seal as before. The first conduit may be coaxial with the product space.

A second fluid pathway may be similarly formed, extending from the proximal end of the product space to an inlet port or breather port, optionally via a filter component, as described above.

A third fluid pathway may be similarly formed, extending from a junction with the product space part way along the product space, for example from adjacent to a region where the boundary between concentrated cell product and supernatant liquid may form following centrifugation.

A fourth fluid pathway may be provided, similarly formed, extending from the distal end of the product space or from a junction with the first fluid pathway adjacent to the distal end of the product space, leading via a fourth fluid conduit to a further port.

The layered assembly may be housed within a rigid housing in order to give mechanical strength and to control the conformation that the layered assembly may adopt in use. The rigid housing may be a close fit to the layered assembly, and the layered assembly may be bonded or clipped to it. The housing may be adapted to be accommodated within a conventional commercially available centrifuge. The housing may instead be adapted to be accommodated within a modified centrifuge as will be described later. The housing may inter-fit with ports on the layered assembly to retain the ports in known locations or configurations, which might otherwise not be maintained owing to the less rigid or even flexible nature of the layered assembly, and may present the ports to inter-fitting apparatus to effect fluid flow through the ports.

The layered assembly may be fabricated using methods as known in the art for blood bag technology, using similar materials. Flexible layers may be RF-welded with rigid spacer components between them. The pattern of product space, conduits and port components may be defined in an RF sealing tool and a complete series of bonds might be formed in a single pass.

The cell product package may be filled individually by introducing a cell product through one of the ports, via a fluid pathway through a conduit, to the product space. Clearly the fluid pathway for filling should not have a filter component within it, so the first fluid pathway, which may also be used for withdrawal of concentrated cell product, may be used to fill the package. A further fluid pathway, optionally via a further fluid conduit, may be provided to allow filling of the product space. The port opening to the fluid pathway to be used may have features allowing ready interconnect of a cell product supply container to the package. When filled, RF sealing may be used to seal the fluid pathway used for filling. This may also be done for any filled reservoirs present in the package. A fluid pathway used for filling of cell product may be provided with a first port that communicates with the fluid pathway via a filter, and a second port that communicates without a filter, the second port for use in filling and sealed thereafter, so allowing filling with cell product while bypassing the filter. The cell product package may be filled before the layered assembly is mounted in the housing or afterwards. The location of the RF seal point(s) may be chosen with regard to the housing design. One or more locations associated with the housing may be provided to indicate the position for sealing, to facilitate sealing, or to allow access of a seal tool to the layered assembly for sealing.

A number of cell product packages may be filled in a common operation by providing a multi-package assembly that may comprise more than one cell product package on a common substrate. The cell packages may each have a fluid pathway in communication with one or more common fluid conduits on the substrate that may function as common filling or emptying lines for the packages. For example, the cell product filling pathways for two or more cell product packages may open to a common conduit on the substrate, allowing the two or more cell product packages to be filled from the common line. The common line may terminate in a septum, valve, or port to allow connection of a cell product container as before.

If further reservoirs are present in the cell product packages, for example for saline, then these reservoirs may be in fluid communication with a second common fluid fill line and may be filled in common. If more than one common fill line is present then a situation may arise where one fill line has to cross another one in order for both fill lines to reach all the packages on a substrate. In this case, a first fill line may be provided on a first face of the substrate, for example the face on which the product spaces have their enlarged profile, and a second fill line may be provided on the second face.

The multi-package assembly may be filled from a the one or more common lines, and then the fluid pathways to each individual package may be sealed to create a number of fluidically separate, sealed cell product packages formed on a common substrate. The fill lines may be emptied prior to sealing, for example by compression, either to empty the fill lines into the product spaces or saline reservoirs of the product packages, or to a waste reservoir. The fill lines may be sealed closed. The cell product package filling fluid pathways and fill lines may be sealed sequentially or at a common time. The multi-package assembly may be further processed to cut out individual cell product packages.

The multi-package assembly may be adapted to inter-fit with further components, for example a housing assembly comprising multiple housings, preferably aligned so that the processed multi-package assembly will inter-fit simply with the housing assembly with each cell product package layered assembly within its own housing. The housing assembly may then be lidded with a corresponding lid assembly, the housing and lid sealed together, and the multiple completed cell product packages may then be separated into single units, ready filled.

Such a common fill and finish process will have considerable advantage in situations where multiple aliquots of a cell product are needed from a common supply, such as in standard supply of cell cultures for research and diagnostic purposes, for distribution of allogeneic cell therapies, or in autologous cell therapy situations where multiple doses of cells are to be produced and packaged at the same time. In situations where volume reduction by centrifugation of the package is not required, the housing might be omitted.

In a further aspect the invention provides an apparatus for filling and sealing a cell product package comprising a layered assembly, comprising:

A platen to receive a layered assembly substrate

A clamp means interoperable with the platen and a layered assembly to compress one or more regions of the layered assembly

An sealing means, interoperable with the clamp means, the layered assembly and the platen

A control unit adapted to control the operation of the apparatus.

The apparatus may additionally comprise a filling means, for example a syringe or pump means.

The apparatus may additionally comprise a temperature control means to control the temperature of the platen, in order to control the temperature of the layered assembly and its contents.

In use, the multi-package assembly is placed on the platen and the clamp means may be placed in a first configuration to hold the layered assembly in place. A cell product container may then be connected to the inlet port of the common cell product fill line. Cell product is then flowed into the fill line by means such as compression, pressure, movement of a plunger if the container is of syringe or piston/cylinder form. Cell product packages are filled from the common fill line and any air that needs to be vented from the product spaces is vented through breathers associated with the product packages. Once the packages are filled, the clamp means may be placed in a second configuration at which the common fill line is compressed to substantially empty it—emptying may be in the direction of the cell product packages. The sealing means may then seal the fill conduits leading to the product packages, leaving these filled, sealed and isolated. Optionally, the apparatus may comprise cutting means that cut out the multi-package assembly substrate to a desired pattern. Finally the clamp means may be removed and the filled array of cell product packages removed from the apparatus. The apparatus may comprise more than one clamp means in order to provide the clamping action. The clamp means may be combined with the sealing means. The sealing means may comprise for example an RF sealing means, a heat sealing means or an ultrasonic sealing means.

In a further aspect the invention provides a kit for packaging a cell product comprising:

One or more cell product packages comprising a layered assembly

Apparatus for filling, sealing and optionally further finishing the cell product packages

In a further aspect the invention provides a kit for packaging a cell product into more than one cell product package in a common operation or sequence of operations comprising:

A multi-package assembly comprising two or more cell product packages formed on a common substrate

Apparatus for filling, sealing and optionally further finishing the cell product packages, resulting in a filled array of cell product packages on a common substrate.

The kit may optionally further include housing and lid components to provide an outer rigid housing for individual cell product package bag assemblies once that have been separated from the filled multi-package assembly.

The invention further provides a system for packaging and distribution of cell products comprising:

One or more cell product packages as above

Apparatus for filling, sealing and optionally further finishing the cell product packages

Housing and lid components to provide protection for the filled cell product package layered assembly

A controlled environment shipper as described above

And optionally a cell product distribution and tracking system as described above

In a further aspect the invention provides a method of packaging and distributing a cell product comprising the steps of:

Providing a system for packaging cell products as described above

Placing the multi-package assembly into a location forming part of a fill and seal apparatus

Flowing cell product into the common fill line causing one or more cell product packages to be filled from the fill line

Activating a sealing means to seal the fill conduits leading to the product packages so as to leave these filled, sealed and isolated.

Optionally, the fill and seal apparatus may move a clamp means to a configuration in which the common fill line is compressed to substantially empty it

Optionally, the fill and seal apparatus may comprise cutting means that cut out the multi-package assembly substrate to a desired pattern.

In a further aspect the invention provides an apparatus inter-operable with the cell product package to achieve processing of the cell product within the package, the apparatus comprising:

A centrifuge adapted to accept the cell product package and to separate cells from the cell product within the cell product package

A first flow system interoperable with the cell product package to remove fluid from the package

A second flow system interoperable with the cell package to add fluid to the package

The first flow system may comprise a first syringe, or a first pump system, that inter-fits with a port on the package to withdraw fluid from the package.

The second flow system may comprise a second syringe, or a second pump system, that inter-fits with a port on the package to introduce fluid into the package.

The first and second flow systems may form part of an apparatus adapted to supply fluid to the cell product package or to receive fluid from it. The apparatus may comprise a control system and one or more pumps or pressure sources to achieve these functions.

In the case that the package comprises reservoirs between which liquid may be moved, the first and second flow systems may provide positive and/or negative pressure gas supplies in order to effect movement of fluids in the cell product package, without liquid passing the interface between the first and second flow systems and the package.

In a further aspect the invention provides a method for the processing of a cell product which comprises the steps of:

Providing a cell product package and a cell product processing apparatus as described above

Centrifuging the cell product package with its distal end facing radially outwards so as to effect separation of cells from the cell product into the tapered distal end of the cell product package

Applying negative fluid pressure via a first flow system at a first port on the package, the port in fluid communication with a fluid pathway provided on the package, so as to remove liquid from the product space while partly or substantially leaving concentrated cell product in place in the taper region

Connecting a patient administration device into fluid communication with a fluid pathway provided on the cell product package and causing cell product to move from the package via the first fluid pathway into the patient administration device.

The method may further comprise one or more of the steps of:

Applying positive fluid pressure via a second flow system at a second port on the package, the port being in fluid communication with a fluid pathway provided on the package, so as to cause a liquid to flow through via the fourth fluid pathway into a region in which concentrated cell product resides, so causing the concentrated cell product to become partly or substantially re-suspended in the liquid.

Centrifuging the cell product package a second time in order to effect separation of cells from the re-suspended cell product towards the tapered distal end of the cell package.

These steps may bring about the following desirable effects: a first medium, for example a culture medium in which cells are transported, is at least partially removed from the product and replaced by a second medium, for example a medium suitable for administration to a patient; the cell product is reduced in volume from a first volume in which the cell product is distributed to a second, smaller volume in which it may be administered to a patient; the concentrated cell product is re-suspended in a liquid which is suitable for administration to a patient, for example sterile saline; the re-suspended cell product is concentrated by the second centrifuging step to a desired concentration for administration; the cell product is loaded into an administration device using a dedicated fluid pathway communicating with the distal end of the tapered region so as to achieve efficiency in the loading process.

The method may involve the removal of waste media or supernatant liquid from the package into the first flow system and the supply of saline from the second flow system to the package. The method may involve application of gas pressure and flow of gas to/from the first and second flow systems in order to effect movement of liquids on the package from a first location or reservoir to a second. For example, the method may involve application of negative gas pressure in a flow pathway via a first port to effect movement of supernatant from the product space into a waste reservoir on the package. The method may involve application of a positive gas pressure in the a flow pathway via a second port to effect movement of saline from a reservoir into the product space in order to re-suspend a concentrated cell product.

The steps of: re-suspending the cell product in new liquid by means of flow though a fluid pathway to the distal end of the product space; centrifuging to concentrate the cells; removing supernatant liquid by means of flow through a third fluid pathway may be repeated so as to wash the cells of remaining transport media.

In a further aspect the invention provides a method for distributing cell product to a point of care and processing the product at the point of care to prepare it for administration to a patient, comprising the steps of:

Providing a cell product package substantially as described herein

Filling the cell product package with cell product in a first medium at a first cell concentration chosen to be appropriate for transport

Sealing the cell product package and transporting it to the point of care

At the point of care, using the method described above for processing the cell product, so providing within the package a processed cell product

Loading the processed cell product into a patient administration device connected to the package.

The method may comprise additional steps including:

Reading/writing data to/from a data tag forming part of the cell product package.

Associating data from the tag on the cell product package with further data in a remote database.

In an embodiment of the invention the biological material handling device comprises a cell product package adapted to contain, transport and to enable the processing of a cell product within the package in the apparatus and according to the method of the invention. Therefore the invention provides embodiments as follows, each of which may have the various features described above to adapt them for use in the apparatus and system of the invention, for example data tags and adaptation for sensors to be provided as part of the package.

FIG. 7a shows an embodiment of a cell product package 10 according to the invention, comprising a housing 12, a product space 14, a closure 15 such as a cap, a first fluidic conduit 16 comprising in this embodiment a septum means 18. The fluidic pathway from the distal end of the tapered region to the exterior is closed firstly by the septum and may also be closed by one or both of a seal cap 20 and a tear-off seal 22. In use, a cell suspension within the product space 14 may be concentrated into the tapering region by means of centrifugation, and then may be withdrawn by a fluid transfer means such as a syringe from the tapered region. The advantages of having the point of withdrawal arranged at the distal end of the device include: the use of a short, fine gauge needle to withdraw the product, which is advantageous if the volume of the concentrated product is small; avoiding the need for interior conduits or long needles if the product is to be accessed from the proximal end of the device: this is particularly useful if the product is to be concentrated from a dilute suspension, in which case the product space 14 is preferably long compared with the tapered region 16. The device may further comprise a breather means 24 adapted to equalise pressure within the product space as material is withdrawn, allowing withdrawal to be achieved without removing the closure 15. The breather means may open to a point at or near the proximal end of the product space, for example being provided as part of the closure 15 or as part of the housing 14 as will be described later for further embodiments. The breather means may comprise a filter such as a 0.22 um sterile filter within the fluid pathway through it in order to maintain sterility of the interior of the product space. The embodiment in FIG. 7a may have dimensions as described for further embodiments below.

FIG. 7b shows an embodiment of an apparatus according to the invention in which a cell product package 100 comprises a housing 102 that defines a product space 104 adapted to contain a cell product, the product space having an elongated form with a long axis and a tapering profile defining a tapered region 106 at the distal end 108 of the product space, the housing being adapted for use in centrifugation;

A first fluid conduit 110 defining a fluid pathway 112 that opens to the interior of the product space at or adjacent to the distal end;

A closure device 114 closing the first fluid conduit, the closure device being openable to allow fluid to flow along the first fluid conduit;

A second fluid conduit 116 defining a second fluid pathway 118 that opens to the interior of the product space at or adjacent to the proximal end;

Breather means 120 in fluid communication with the second fluid pathway, adapted to allow equalisation of pressure in the product space when fluid is flowed along the first fluid pathway.

The housing may be of rigid, semi-rigid or flexible form.

The product space may be of any appropriate cross sectional shape, for example substantially one of: circular, semi-circular, trapezoid or rectangular.

The product space may be of dimensions appropriate to the volume of cell product to be contained. The volume within the housing may be for example in the range 1000 to 100, 100 to 10, 10 to 1 or 1 to 0.1 ml. The housing may be narrower in cross section than it is long, with the aspect ratio of length to typical dimension perpendicular to the axis of the housing for example in the range 1:1 to 20:1.

The housing may have a length along its axis in the range of 20 to 10 cm, 10 to 5 cm, or less than 5 cm and a dimension perpendicular to the axis in the range of 5 to 3 cm, 3 to 1 cm or less than 1 cm.

The housing may be adapted for centrifugation by comprising features such as: appropriate material; strength and fabrication method appropriate for the stresses encountered in centrifugation; axial symmetry in shape or distribution of mass to promote uniform behaviour during centrifugation; dimensions suitable to fit within a standard commercially-available centrifuge.

The taper may be uniform, for example conical or semi-conical, or non-uniform, for example with a cross-sectional dimension decreasing in one or more steps. The taper may comprise a change in cross-sectional shape, for example from trapezoidal to circular or semi-circular.

The taper is adapted to facilitate concentration of cells from a cell product comprising a suspension of cells towards the distal end of the product space, leaving a supernatant liquid above the concentration zone. The angle, or step size(s) of the taper are adapted to give a chosen degree of concentration.

The first fluid conduit may be adapted to allow flow of a cell product along the first fluid pathway for example to: load a cell product into the product space; to remove a cell product from the product space; to flow a liquid medium into the product space; to withdraw a liquid medium from the product space; to withdraw a concentrated cell product from the product space, for example into a patient administration device such as a syringe, syringe needle or catheter.

The first fluid conduit may extend within the housing substantially parallel to the axis of the housing and have a first opening adjacent to the narrow end of the taper, and a second opening at the proximal end of the housing. The first fluid conduit may be substantially coaxial with the product space or may lie away from the axis, for example adjacent to a wall of the product space.

FIG. 8 shows a further embodiment of a device according to the invention.

As shown in FIG. 8 the first fluid conduit 110 may be formed within an extension 122 to the housing beyond the taper and may be coaxial with the product space. The first fluid pathway may open to the product space at, or adjacent to, the narrowest part of the taper. This arrangement may facilitate removal of cell product along the first fluid pathway towards a patient administration device such as a syringe needle.

The first fluid conduit may be closed by a closure device 124 such as a septum or valve that may be pierced or opened by a tool such as a tube, projecting plug portion, pipette tip, syringe, needle or other form of fluid connection means. The closure device may be adapted to re-seal after opening. The closure means may be a septum that may be pierced by a patient administration device such as a syringe needle or catheter in order to withdraw cell product from the tapered section of the product space.

The closure device may be covered by an outer sterile closure 126 such as a snap-off cap or a tear-off strip so ensuring sterility of the closure device outer surface until access to the cell product is needed.

The first fluid pathway may be adapted to supply liquid medium to the product space, in particular to supply it in the vicinity of the tapered portion. Dimensions of the first fluid conduit may be chosen according to whether liquid medium, cell product or concentrated cell product are to pass along it.

The second fluid conduit may be adapted to allow flow of fluid into the product space to equalise wholly or partially changes of pressure in the product space when a cell product is withdrawn or introduced via the first fluid conduit.

The second fluid pathway may be closed by a breather means such as a porous membrane or a valve in order to allow air to flow along the second fluid pathway when a cell product is withdrawn or introduced via the first fluid conduit.

The second fluid conduit may be adapted for use in filling the product space with cell product, air from the product space being expelled via the first fluid pathway. The first fluid pathway may be provided with a breather means as above to facilitate this.

The cell product package may comprise a third fluid conduit 130 forming a third fluid pathway 132 extending from a point within the product space to a point at or adjacent to the proximal end of the housing. The third fluid conduit may open at a region 134 in the product housing adjacent to the taper, for example at a region within the taper adjacent to the level at which the boundary between the concentrated cell product and the supernatant liquid will form following centrifugation. Supernatant liquid may be withdrawn along the third fluid conduit leaving concentrated cell product in situ in the taper region.

The third fluid conduit 130 may extend within the housing substantially parallel to the axis, and may be coaxial or nearly so with the axis. It may be located away from the axis, for example adjacent to a wall of the product space.

The cell product package may comprise a fourth fluid conduit 160 forming a fourth fluid pathway 162 extending from a point adjacent to the distal end of the product space or from a junction with the first fluid pathway adjacent to the distal end of the product space, leading via the fourth fluid conduit to a further port 164. Such a fourth fluid pathway may be used for example to introduce fluid into a region adjacent to the distal end of the product space without that fluid flowing along the first fluid pathway, which may then be reserved for flow of cellular product and/or concentrated cellular product.

The fluid conduits may be formed in a number of ways known in the art. They may comprise tubular structures formed or mounted within the housing. For example tubular sections extending within the product space to open at the appropriate region. The tubes may be formed from plastic tubing sections, metal section such as syringe tubing and may be rigid or flexible. The tubes may be formed as part of one or more moulded components mounted on or formed as part of the housing. More than one fluid conduit may be formed as part of a component, for example a dual lumen tube or moulding.

One or more fluid pathways may be closed by further closure means, for example septa or valves, and outer sterile caps 154 or tear-off strips 156 that might cover more openings to more than one fluid pathway in common. One or more of the fluid pathways may comprise a filter 136, such as a 0.22 um pore filter, to prevent contamination of the interior of the device. The filter(s) may be provided as a substantially planar component through which the fluid pathway passes; the planar component may be located within the housing at the proximal end.

The housing may be formed for example by moulding and may comprise a single component or more than one. The housing may comprise a tubular component 140 having a taper at the distal end and a cap or closure component 142 that seals to the tubular component to form a closed product space. One or more fluid conduits may extend from the cap component into the tubular component. One or more fluid conduits may be formed as part of the tubular component or attached to it. One or more fluid conduits may be formed partially within the cap component, and the fluid pathways may pass through the cap component from a first, inner face 144 in contact with the product space to a second outer face 146. One or more filter component(s) 136 may be provided within or mounted on the cap component to introduce a filter element into the fluid pathway(s).

One or more fluid pathways may extend to openings or ports 148, 150, 160 in the cap that may be adapted to connect to further fluid components, for example, fluid connections, syringes, needles. Ports may be for example adapted to accept standard fluid connections such as Luer fittings. Ports may be closed by valves, such as flap valves or septum valves comprising a slit, as known in the art. One or more fluid pathways, for example the second fluid pathway, may extend to a breather port 152 opening to the second surface 146 of the cap.

The ports and/or the cap may be closed by a sterile closure such as a snap-off cap 154 or a tear-off strip 156 to maintain sterility of the ports and the fluid pathway until the time of use.

FIG. 9 shows a further embodiment of a cell product package according to the invention with common features having the same numbers as in FIGS. 1 and 2.

As shown in FIG. 9, a cell product package 100 may further comprise one or more reservoirs that may form part of one or more fluid pathways. Such reservoirs may comprise reservoirs for cell culture medium, saline or other medium suitable for suspension of cell product for administration to patients, wash or waste reservoirs.

A reservoir 170 for waste solutions may be provided as part of the second or, as shown in FIG. 9, the third fluid pathways.

A reservoir 172 for saline solution may be provided as part of a fourth fluid pathway.

A reservoir for further fluids, for example a cell culture medium, may be provided as part of the first fluid pathway or a further fluid pathway, for example a fifth fluid pathway provided as part of the cell product package.

One or more reservoirs may be filled at the point of filling cell product into the product space, for example the saline reservoir, and the cell product package sealed and transported containing the saline. The waste reservoir may be empty following the point of filling or may be partially filled.

The device may be adapted so that once filled, only cell product is removed from the device: other liquids are retained within it. Actuation of flow of fluids within the device may be by gas pressure at the ports leading to the fluid pathways, the gas pressure acting to move liquids within the device.

For example, in FIG. 9 the third fluid pathway 132 is shown as comprising a waste reservoir 170 and terminating at a port 150. In use port 150 may inter-fit with a connector 174 defining a fluid pathway 176 to an actuating device such as a pump or a pressure source, for example a syringe. Negative pressure in fluid pathway 176 relative to atmospheric will tend to draw liquid along pathway 132 into the waste reservoir. Breather and/or filter 136 may act to allow gas to flow through it while preventing liquid from exiting along fluid pathway 176, so ensuring liquid is contained within the package 100. Similarly the fourth fluid pathway 162 is shown as comprising saline reservoir 172 and terminating at a port 164. In use port 164 may inter-fit with a connector 178 defining a further fluid pathway 180 to an actuating device such as a pump or a pressure source, for example a syringe. Positive pressure in fluid pathway 180 relative to atmospheric will tend to move liquid along pathway 132 out from the saline reservoir into the distal end 108 of the product space. As before a breather and/or filter 136 may act to allow gas to flow through it while preventing liquids from exiting along fluid pathway 180, so ensuring liquid is contained within the package 100.

In this way the device may operate as a closed system, with no open interfaces across which liquids will travel, until the point at which concentrated cell product is withdrawn from the device via the first fluid pathway 112, shown as closed by septum 124 until the septum is pierced by patient administration means 182.

The housing may be substantially circular in cross-section, for example in the form of a moulded tube. The housing may be substantially rectangular, and may be assembled from a layered assembly of substantially planar components with recesses or through channels within them defining walls of the product space, conduits and reservoirs where present. For example, the housing may be formed from a body part comprising recesses and/or channels with one or more lid components mounted on one or both faces. The housing may be formed from a layered assembly comprising a profiled first component comprising recesses and/or channels bonded to a substantially planar component to close the recesses and/or channels.

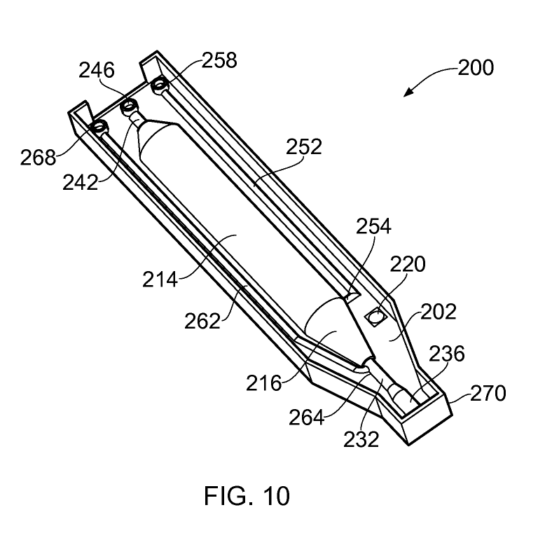

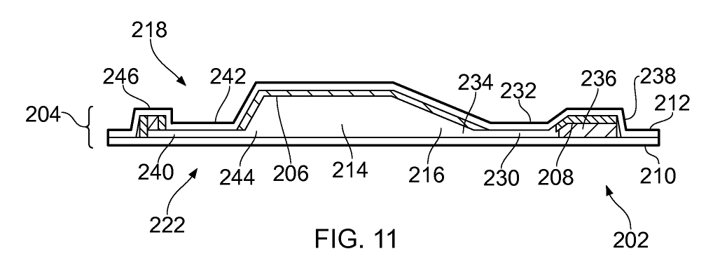

FIG. 10 shows a three-quarter view of a cell product package according to the invention and FIG. 11 a view in cross sectional along the axis of the device in figure

As shown in FIGS. 10 and 11, a cell product package 200 may comprise a layered assembly 202 comprising at last one flexible component 204 and one or more rigid components. The flexible component(s) 204 may be largely or only partially flexible, and may comprise material and use fabrication techniques used in fluid bag technology as known in the art. The flexible component may enclose or be bonded to one or more rigid components 206, 208 so as to provide an assembly that has flexible regions and less flexible, or rigid, regions. The layered assembly may comprise one layer of flexible material bonded to a rigid component, or more than one layer of flexible material, having as least two layers 210, 212 bonded together to enclose a rigid component. The layered assembly may comprise a product space 214 having an elongated form as before, with a taper region 216 at the distal end. The product space may be defined by a rigid or semi-rigid spacer component 206 bonded between two layers of flexible material, defining a rigid space within the layered assembly with an enlarged profile on a first face 218 of the layered assembly. The spacer component may have a larger cross sectional dimension at a proximal end and a smaller at the distal end, so producing a tapered space when the components are bonded together. The spacer component may be adapted to produce for example a semi-circular, polygonal or trapezoidal cross sectional shape. Dimensions of the product space may be as before.

The cell product packages preferably comprise a data tag 220, for example an RFID data tag as described herein, which might be included in the package during the assembly process, for example embedded in the package and sealed in place under one of the flexible layers, or may be mounted on or within the outer housing 270.

The product package may comprise one or more fluid conduits defining fluid pathways. The one or more fluid conduits may be defined as passages between layers of flexible material, or may comprise tubular or non-tubular forming elements to facilitate definition of the conduits. The conduits may have the same functions as described above. One or more conduits may be comprise insert components that may define or retain for example a septum, a valve, such as a septum comprising a preformed slit; a breather component, a filter or other component in a fluid pathway.

Conduits may have an enlarged profile on a first face 218 of the layered assembly and a flatter profile on the other 222. The layered assembly may comprise one, two, three or more layers of flexible material bonded together. Conduits may be formed with their enlarged profile on the first face of the layered assembly in common with the product space or on the other, second face. For example, in a three layer layered assembly comprising a product space formed between layers 1 and 2, conduits may be formed between layers 1 and 2 or between layers 2 and 3, or both. Vias may be provided linking conduits on the first face and on the second face. Conduits on the first face may cross conduits on the second face without joining.

A first fluid pathway 230 may be formed as a first conduit 228 between two layers of bonded flexible material, the conduit extending from the distal end 230 of the product space, and terminating in a component 232 comprising a septum adapted or withdrawal of cell product following centrifugation. The septum might be sealed with a sterile seal 234, which may be formed by the layered assembly process itself. The first conduit may be coaxial with the product space.

A second fluid pathway 240 may be similarly formed by a second conduit 242, extending from the proximal end 244 of the product space to an inlet port 246 or breather port, optionally via a filter component and/or septum or valve, as described above.

A third fluid pathway 250 may be provided, similarly formed by a third conduit 252, extending from a junction 254 with the product space part way along the product space, for example from adjacent to a region 256 where the boundary between concentrated cell product and supernatant liquid may form following centrifugation, to an inlet port 258 or breather port, optionally via a filter component and/or septum or valve.

A fourth fluid pathway 260 may be provided, similarly formed by a fourth conduit 262, extending from adjacent to the distal end 234 of the product space or from a junction 264 with the first fluid pathway adjacent to the distal end of the product space, leading via a fourth fluid conduit to a further inlet port or breather port 268, optionally via a filter component and/or septum or valve.

The layered assembly 202 may be housed within a rigid housing 270 in order to give mechanical strength and to control the conformation that the layered assembly may adopt in use. The rigid housing may be a close fit to the layered assembly, and the layered assembly may be bonded or clipped to it. The housing may be adapted to be accommodated within a conventional commercially available centrifuge. The housing may instead be adapted to be accommodated within a modified centrifuge as will be described later. The housing may inter-fit with ports on the layered assembly to retain the ports in known locations or configurations, which might otherwise not be maintained owing to the flexible nature of the layered assembly, and may present the ports to inter-fitting apparatus to effect fluid flow through the ports.

The layered assembly may be fabricated using methods as known in the art for blood bag technology, using similar materials. Flexible layers may be RF-welded with rigid spacer components between them. The pattern of product space, conduits and port components may be defined in an RF sealing tool and a complete series of bonds might be formed in a single pass.

The cell product package may alternatively be assembled from a layered assembly of substantially planar rigid or semi-rigid components with recesses or through channels within them defining walls of the product space, conduits and reservoirs where present. For example, the housing may be formed from a body part comprising recesses and/or channels with one or more lid components mounted on one or both faces. The housing may be formed from a layered assembly comprising a profiled first component comprising recesses and/or channels bonded to a substantially planar component to close the recesses and/or channels.

The cell product package may be filled individually by introducing a cell product through one of the ports, via a fluid pathway through a conduit, to the product space. Clearly the fluid pathway for filling should not have a filter component within it, so the first fluid pathway, which may also be used for withdrawal of concentrated cell product, may be used to fill the package. A further fluid pathway, optionally via a further fluid conduit, may be provided to allow filling of the product space. The port opening to the fluid pathway to be used may have features allowing ready interconnect of a cell product supply container to the package. When filled, RF sealing may be used to seal the fluid pathway used for filling. This may also be done for any filled reservoirs present in the package. A fluid pathway used for filling of cell product may be provided with a first port that communicates with the fluid pathway via a filter, and a second port that communicates without a filter, the second port for use in filling and sealed thereafter, so allowing filling with cell product while bypassing the filter. The cell product package may be filled before the layered assembly is mounted in the housing or afterwards. The location of the RF seal point(s) may be chosen with regard to the housing design. One or more locations associated with the housing may be provided to indicate the position for sealing, to facilitate sealing, or to allow access of a seal tool to the layered assembly for sealing.

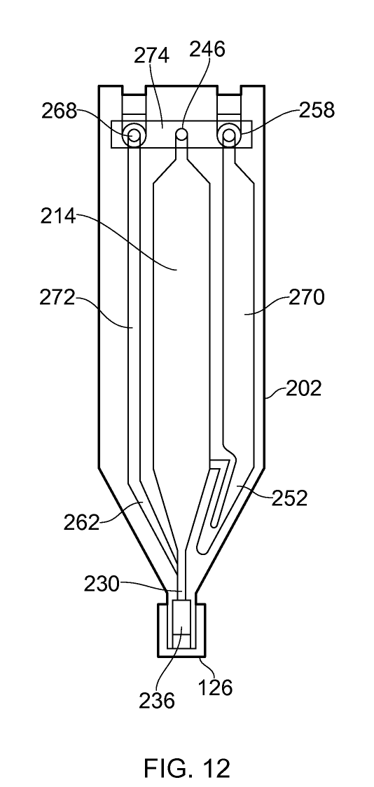

FIG. 12 shows a further embodiment of a cell product package according to the invention, in which reservoirs are provided as part of one or more fluid pathways.

For example, in FIG. 12 the third fluid pathway 252 is shown as comprising waste reservoir 270 and terminating at a port 258. In use port 258 may inter-fit with a connector defining a fluid pathway to an actuating device such as a pump or a pressure source, for example a syringe, as described for the embodiment in FIG. 9. Negative pressure in the fluid pathway relative to atmospheric will tend to draw liquid along pathway 252 into the waste reservoir. Breather or filter 274 may act to allow gas to flow through it while preventing liquid from exiting along the fluid pathway, so ensuring liquid is contained within the package. Similarly the fourth fluid pathway 262 is shown as comprising saline reservoir 272 and terminating at a port 268. In use port 268 may inter-fit with a connector defining a further fluid pathway to an actuating device such as a pump or a pressure source, for example a syringe. Positive pressure in the fluid pathway relative to atmospheric will tend to move liquid along pathway 262 out from the saline reservoir into the distal end of the product space. As before a breather or filter 274 may act to allow gas to flow through it while preventing liquids from exiting along the fluid pathway, so ensuring liquid is contained within the package.

FIGS. 7-10 shows stages in the assembly of a further embodiment of a cell product package according to the invention.

As shown in FIG. 13 a number of cell product packages may be filled in a common operation by providing a multi-package assembly 300 that may comprise more than one cell product package 302 on a common substrate 304, comprising at least one flexible layer plus a rigid layer, or two or more flexible layers. The cell packages may each have a fluid pathway 306 in communication with one or more common fluid conduits 310 on the substrate that may function as common filling or emptying lines for the packages. For example, the cell product filling pathways for two or more cell product packages may open to a common conduit on the substrate, allowing the two or more cell product packages to be filled from the common line. The common line may terminate in a septum, valve, or port 312 to allow connection of a cell product source.

If further reservoirs are present in the cell product packages, for example for saline, then these reservoirs may be in fluid communication with a second common fluid fill line and may be filled in common. If more than one common fill line is present then a situation may arise where one fill line has to cross another one in order for both fill lines to reach all the packages on a substrate. In this case, a first fill line may be provided on a first face of the substrate, for example the face on which the product spaces have their enlarged profile, and a second fill line may be provided on the second face.

The multi-package assembly may be filled from the one or more common lines, and then the fluid pathways to each individual package may be sealed as shown in FIG. 14 to create a number of fluidically separate, sealed cell product packages formed on a common substrate. The fill lines may be emptied prior to sealing, for example by compression, either to empty the fill lines into the product spaces or saline reservoirs of the product packages, or to a waste reservoir. The fill lines may be sealed closed as shown as 314 in FIG. 14. The multi-package assembly may be further processed to cut out individual cell product packages.

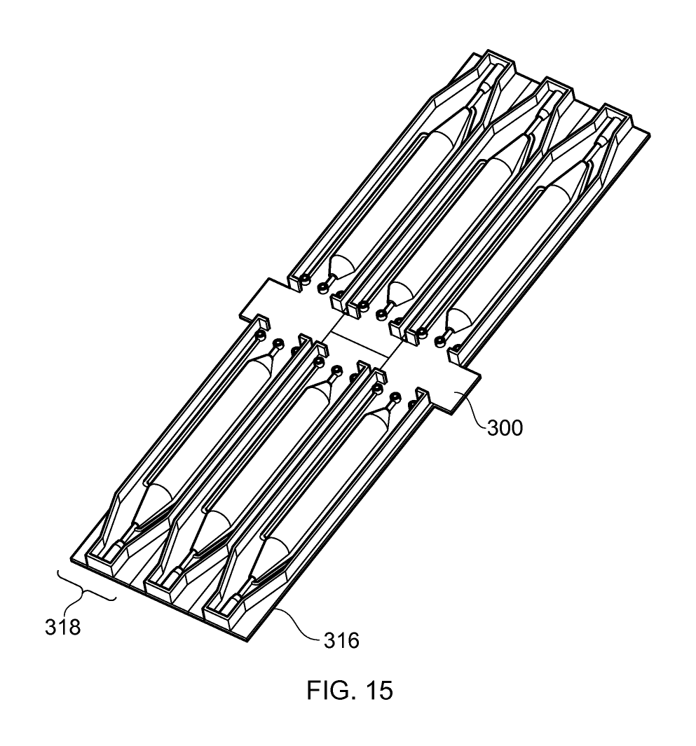

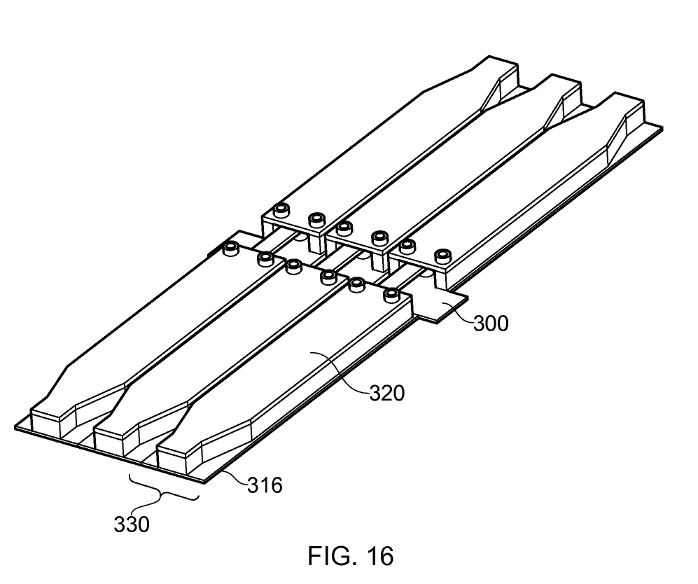

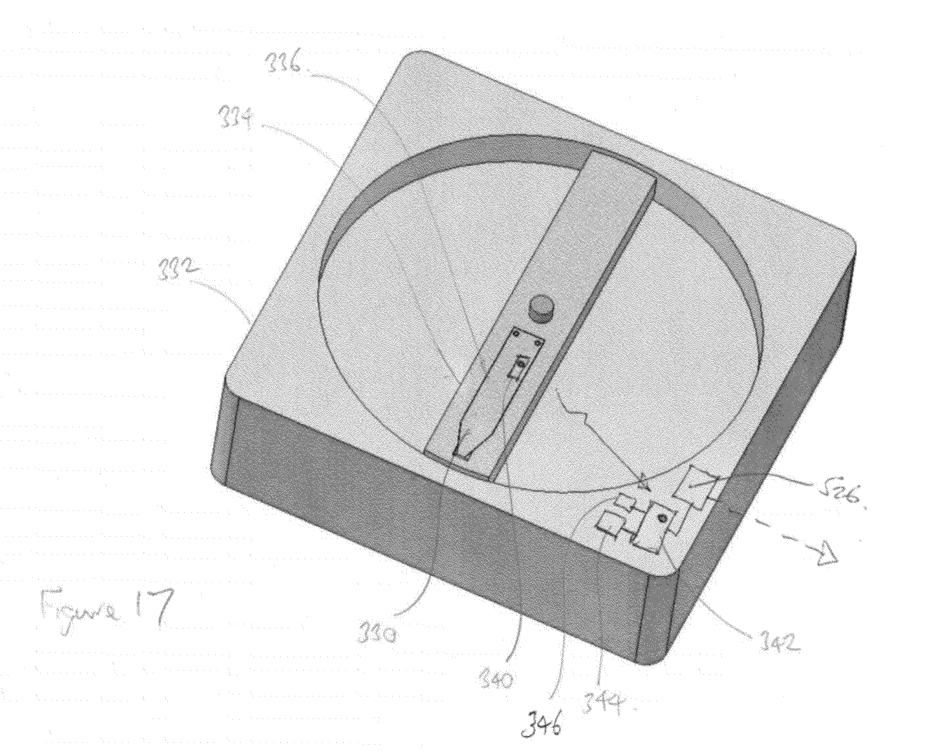

The multi-package assembly may be adapted to inter-fit with further components, for example a housing assembly 316 comprising multiple housings 318 as shown in FIG. 15, preferably aligned so that the processed multi-package assembly will inter-fit simply with the housing assembly with each cell product package layered assembly within its own housing. The housing assembly may then be lidded as shown in FIG. 16 with a corresponding lid assembly 320, the housing and lid sealed together, and the multiple completed cell product packages may then be separated into single units 330, ready filled. Alternatively, the assembly process as shown in the figures may be used to form completed individual packages that may then be filled and sealed individually.

FIG. 17 shows a centrifugation instrument 332 that forms a component apparatus as part of the apparatus and system of the invention and is adapted to centrifuge the cell product package to achieve concentration of cells within the cell product within the product space. In a preferred embodiment the centrifugation instrument comprises a rotor 334 comprising a recess 336 adapted to receive the cell product package. A specific centrifugation instrument arrangement and rotor design is shown here, though in some embodiments the cell product package is adapted to fit a standard centrifuge. In a preferred embodiment the cell product package comprises a data tag 340, preferably adapted to monitor the T/time conditions experienced by the cell product package. The instrument 332 preferably comprises means 342 to read data from the data tag 340. In a preferred embodiment the instrument reads device information comprising command instructions relating to the cell product and acts in accordance with those instructions to process the product within the package. In a further preferred embodiment the instrument comprises local control means 526 and may communicate with further components of the system according to the invention, for example by means of a data system as described before. The instrument may comprise a process initiation control 344, and may comprise an indicator means 346 such as a go/no-go indicator to indicate whether processing has completed in regulatory compliance. The instrument 332 may comprise any or all of the further features as described above.

What is claimed is:

1. Apparatus for monitoring multiple-stage processing of a biological material, comprising:
a data tag attached to a biological material handling device containing the biological material, wherein the data tag provides information of the biological material handling device during the processing of the biological material;
one or more sensors attached to a component apparatus usable to process the biological material, wherein the one or more sensors monitor a condition associated with the biological material handling device during the processing of the biological material;
a data system adapted to
collect data of the biological material handling device from both the data tag and the sensors;
associate the data read from the tag and the sensors with data stored in a database related to the processing of the biological material;
analyze the data and generate report on regulatory compliance of the processing of the biological material.

2. Apparatus for processing a biological material through a multiple-stage process, comprising:
a biological material handling device containing the biological material, wherein the device has a data tag attached to the device for providing information of the device during the multiple-stage process;
a component apparatus usable to process the biological material, wherein component apparatus has one or more sensors attached to the component apparatus for monitoring a condition associated with the biological material handling device during the multiple-stage process;
a data system adapted to collect data of the biological material handling device from both the data tag and the sensors;
associate the data read from the tag and the sensors with data stored in a database related to the multiple-stage process;
analyze the data and generate report on regulatory compliance of the processing of the biological material.
